(19) United States
(12) Patent Application Publication
(10) Pub. No.: US 2025/0283166 A1
(43) Pub. Date: Sep. 11, 2025

(54) METHODS AND COMPOSITIONS FOR IDENTIFYING DNA ABERRATIONS

(71) Applicant: Inguran, LLC, Navasota, TX (US)

(72) Inventors: Thomas B. Gilligan, College Station, TX (US); Kenneth Michael Evans, College Station, TX (US); Daniela do Amaral Grossi, Portland, ME (US); Yuri Utsunomiya, São Paulo (BR); Adam Utsunomiya, São Paulo (BR); Pablo Ross, Navasota, TX (US); Nader Deeb, Navasota, TX (US); Vinicius Henrique da Silva, São Paulo (BR)

(73) Assignee: Inguran, LLC, Navasota, TX (US)

(21) Appl. No.: 19/216,435

(22) Filed: May 22, 2025

Related U.S. Application Data
(63) Continuation-in-part of application No. 16/859,783, filed on Apr. 27, 2020.
Application No. 16/859,783 is a continuation-in-part of application No. 16/379,456, filed on Apr. 9, 2019.
(60) Provisional application No. 62/655,040, filed on Apr. 9, 2018.
(60) Provisional application No. 62/673,668, filed on May 18, 2018.

(51) Int. Cl. C12Q 1/6869; C12Q 1/6883; G01N 1/30

(52) U.S. Cl. CPC C12Q 1/6869; C12Q 1/6883; G01N 1/30; C12Q 2600/156

(57) ABSTRACT

The invention consists of methods and compositions for detecting the presence or absence of a DNA aberration by analyzing fluorescence emission characteristics in sperm cells or sperm nuclei, which generally consists of entraining sperm cells or sperm nuclei stained with a DNA selective dye in sheath fluid; exposing the entrained sperm cells or sperm nuclei to electromagnetic radiation; determining a forward fluorescence characteristic and a side fluorescence characteristic of individual events associated with the exposed sperm cells or sperm nuclei; and gating the individual events based on the forward fluorescence characteristic and the side fluorescence characteristic with a criterion.

### REFERENCE TO RELATED APPLICATIONS

This application is a Continuation-in-Part of U.S. patent application Ser. No. 16/859,783 filed on Apr. 27, 2020, which was a Continuation-in-Part of U.S. patent application Ser. No. 16/379,456 filed on Apr. 9, 2019 and claims the benefit of U.S. Provisional Patent Application No. 62/655,040 filed on Apr. 9, 2018 and U.S. Provisional Patent Application No. 62/673,668 filed on May 18, 2018, the entire disclosures of which are incorporated herein by reference.

### BACKGROUND

In mammals, the male (sire) haploid cells (sperm) are presented by ejaculation in very large numbers comprising millions or even billions of cells, each of which carry a somewhat random distribution of autosomes (normally one of a pair of equal sized autosomes) as well as sex chromosomes (X and Y) that normally distribute ½ of the total haplotype of the male into each sperm. In normal healthy males, the sperm population is typically two distinct populations, where variation in total DNA content is based only on the sex chromosome and sorting of such populations of sperm by flow cytometry is a well-established and non-invasive method to preselect sex (create percent of bias of male or female) in mammals.

The use of genetic analysis of individual breeding animals by DNA genotyping or DNA sequencing methods has become a low-cost method to analyze the genetic microstructure and mathematical distribution of haplotype inheritance and is used in large scale to determine the breeding value of an animal. Large stable DNA macrostructural aberrations such as translocations are not analyzed by the above-mentioned microstructural DNA analysis methods, but since they can also lead to poor reproductive performance, such as reduction in litter size (in multiparous species) or pregnancy rates (in uniparous species) by mechanism of early embryonic death, low-cost methods to screen for them are needed.

A common problem, for example in swine breeding, is created when translocations or other stable DNA aberrations are carried forward, particularly in breeding males, by non-lethal inheritance. When the negative phenotype is litter size, the reliance on that phenotypical observation to cull out breeding boars and sows is problematic because progeny carriers are created, and if these animals are then used to generate new breeding males and females they may also be carriers and the problem can remain or even be amplified. For this reason, it has become a common practice to use the long-standing method of karyotype analysis to screen breeding animals, in special boars, prior to their use. Karyotype analysis has the advantage that it can commonly identify large stable changes in DNA macrostructure by reliable and well defined methods, and the advent of computer assisted image analysis has made karyotype analysis routine. Nonetheless, karyotype analysis typically requires handling of fresh specimens (mainly blood), the cultivation of clonal cell lines in defined but artificial media and the analysis of diploid somatic cells as a proxy to direct analysis of germ line cells. The process typically takes a minimum of several days, if not even weeks to complete. It is not highly statistically significant, as the number of cells analyzed is small. There are also limitations in the threshold of percent of DNA change that can be determined. Furthermore, karyotype analysis does not measure any abnormal effects in spermatogenesis that might lead to materially important changes in DNA distribution within sperm populations (gamete aneuploidy).

Additionally, in species such as bovines where artificial insemination is used extensively but conception rate information is difficult and time consuming to recover, karyotype analysis is not reliable and low-cost enough to be used industrially. The current karyotyping procedures takes approximately 3 weeks. A sample must be shipped by overnight courier and in some cases shipments are delayed, and the sample is lost and unable to be analyzed requiring resampling. Karyotyping is costly and prices are expected to increase with a lack of sufficient facilities to perform karyotyping, with some facilities discontinuing service.

Translocations happen at a rate of 1% in swine—therefore a very high number of tests are normal at a high cost. The effect of a translocated boar's semen is a loss of about 4-5 piglets per litter. One single boar can produce thousands of pigs in a lifetime, so if those litter sizes are smaller there is lost productivity. Because of the problem, which is that approximately ⅓ of the sperm produced by a translocated boar will be non-viable due to reduced amount of DNA, decreased fecundity occurs.

### SUMMARY OF THE INVENTION

One embodiment of the invention encompasses a method of analyzing fluorescence emission characteristics in sperm cells or sperm nuclei (i.e., sperm heads), comprising entraining sperm cells or sperm nuclei stained with a DNA selective dye in sheath fluid; exposing the entrained sperm cells or sperm nuclei to electromagnetic radiation; determining a forward fluorescence characteristic and a side fluorescence characteristic of individual events associated with the exposed sperm cells or sperm nuclei; gating the individual events based on the forward fluorescence characteristic and the side fluorescence characteristic with a criterion; and determining the presence or absence of a DNA aberration from the gated individual events. In a further embodiment, the DNA selective dye is Hoechst 33342. Another embodiment further comprises the step of orienting the entrained sperm cells or sperm nuclei. In another embodiment, the step of exposing the sperm cells or sperm nuclei to electromagnetic radiation comprises exposing the sperm cells or sperm nuclei to a laser beam with modified beam profile. In yet another embodiment, the criterion encompasses a subpopulation of oriented sperm cells or sperm nuclei. In these embodiments, the sperm may comprise sperm cells or sperm nuclei from a first and a second mammalian species. In a further embodiment, determining the presence of a DNA aberration from the gated individual events comprises detecting more than two peaks or modes, or a peak to valley ratio of 80% or less, 70% or less, 60% or less, or 50% or less, on a histogram of fluorescence intensities, or measurable differences in co-efficient of variation in two or more modes. In a yet further embodiment, determining the presence of a DNA aberration from the gated individual events comprises detecting a difference between a peak to valley ratio on a histogram of fluorescence intensities of sperm cells or sperm nuclei from the first mammalian species and a peak to valley ratio on a histogram of fluorescence intensities of sperm cells or sperm nuclei from the second mammalian species or measurable differences in co-efficient of variation between two or more modes created by the first and second mammalian species. Another embodiment of the invention comprises subjecting the entrained sperm cells or sperm nuclei to orienting hydrodynamic forces such as those imparted by an orienting nozzle.

The invention also encompasses a method of analyzing fluorescence emission characteristics in sperm cells or sperm nuclei, comprising entraining sperm cells or sperm nuclei stained with a DNA selective dye in sheath fluid; exposing the entrained sperm cells or sperm nuclei to electromagnetic radiation; determining two or more fluorescence emission characteristics of individual events associated with the exposed sperm cells or sperm nuclei; gating the individual events based on the two or more fluorescence emission characteristics with a first criterion for further processing; gating the further processed individual events with a second criterion; and determining the presence or absence of a DNA aberration from the twice gated individual events. In a further embodiment, the first criterion encompasses a first subpopulation of oriented sperm cells or sperm nuclei. In a yet further embodiment, the second criterion encompasses a second subpopulation of sperm cells or sperm nuclei comprising 25% to 75% of the first subpopulation. In an even further embodiment, the step of determining the presence or absence of DNA aberrations in the sperm cells or sperm nuclei from the twice gated individual events further comprises evaluating a third subpopulation of sperm cells or sperm nuclei within the first subpopulation, wherein the third subpopulation of sperm cells or sperm nuclei excludes the second subpopulation of sperm cells or sperm nuclei. In an additional embodiment, the second criterion encompasses 25% to 75% of sperm around a median value of a fluorescence emission characteristic. In a further embodiment, the step of determining the presence or absence of a DNA aberration in the sperm cells or sperm nuclei from the twice gated individual events further comprises determining the quantity of peaks or modes in a twice gated fluorescence emission characteristic. In an even further embodiment, the step of determining the presence or absence of a DNA aberration in the sperm cells or sperm nuclei from the twice gated individual events further comprises analyzing a coefficient of variation of a fluorescence emission characteristic associated with the first subpopulation of sperm cells or sperm nuclei or the second subpopulation of sperm cells or sperm nuclei. In an additional embodiment, the step of determining the presence or absence of a DNA aberration in the sperm cells or sperm nuclei from the twice gated individual events further comprises generating a first univariate plot based on a fluorescence emission characteristic of the first subpopulation or generating and a second univariate plot based on a fluorescence emission characteristic of the second subpopulation. In a further embodiment, the step of determining the presence or absence of a DNA aberration in the sperm cells or sperm nuclei from the twice gated individual events further comprises analyzing a peak to valley ratio of the first univariate plot or a peak to valley ratio of the second univariate plot or measurable differences in co-efficient of variation between modes in multivariate plots. In an even further embodiment, the method further comprises separating tails or midpieces from the sperm nuclei by centrifugation. In a further embodiment, the DNA selective dye is Hoechst 33342. Another embodiment further comprises the step of orienting the entrained sperm cells or sperm nuclei. In another embodiment, the step of exposing the sperm cells or sperm nuclei to electromagnetic radiation comprises exposing the sperm cells or sperm nuclei to a laser beam with modified beam profile. In these embodiments, the sperm cells or sperm nuclei may comprise sperm cells or sperm nuclei from a first and a second mammalian species. In a further embodiment, determining the presence of a DNA aberration comprises detecting more than two peaks or modes, or a peak to valley ratio of 80% or less, 70% or less, 60% or less, or 50% or less, on a histogram of fluorescence intensities or measurable differences in co-efficient of variation between modes in multivariate plots. In a yet further embodiment, determining the presence of a DNA aberration comprises detecting a difference between a peak to valley ratio on a histogram of fluorescence intensities of sperm cells or sperm nuclei from the first mammalian species and a peak to valley ratio or co-efficient of variation in modes on a histogram of fluorescence intensities of sperm cells or sperm nuclei from the second mammalian species. Another embodiment of the invention comprises subjecting the entrained sperm cells or sperm nuclei to orienting hydrodynamic forces such as those imparted by an orienting nozzle.

The invention is also embodied by a method of analyzing a fluorescence emission characteristic in sperm cells or sperm nuclei, comprising staining the sperm cells or sperm nuclei with a DNA selective dye; entraining the stained sperm cells or sperm nuclei in sheath fluid; exposing the entrained sperm cells or sperm nuclei to electromagnetic radiation; determining a fluorescence emission characteristic of the exposed sperm cells or sperm nuclei; generating a first multivariate plot based on the fluorescence emission characteristic; providing a first gate on the first multivariate plot; generating a second multivariate plot based on the first gate; providing a first sort region on the second multivariate plot encompassing a first subpopulation of sperm and a second sort region on the second multivariate plot encompassing a second subpopulation of sperm; separating the first subpopulation of sperm cells or sperm nuclei and the second subpopulation of sperm cells or sperm nuclei; generating a first univariate plot based on a fluorescence emission characteristic of the separated first subpopulation of sperm cells or sperm nuclei; generating a second univariate plot based on a fluorescence emission characteristic of the separated second subpopulation of sperm cells or sperm nuclei; and determining the presence or absence of a DNA aberration based on the first univariate plot or the second univariate plot. In another embodiment, the method further comprises the step of separating tails or midpieces from the sperm nuclei by centrifugation. In yet another embodiment, the first subpopulation of sperm cells or sperm nuclei comprises 25% to 75% of the sperm population. In an even further embodiment, the second subpopulation of sperm cells or sperm nuclei excludes the first subpopulation of sperm cells or sperm nuclei. In an additional embodiment, the first sort region encompasses the center of the second multivariate plot. In yet another embodiment, the method comprises the step of determining the quantity of peaks or modes of the first univariate plot or the second univariate plot, analyzing a coefficient of variation of the first univariate plot or the second univariate plot, or analyzing a peak to valley ratio of the first univariate plot or the second univariate plot. In a further embodiment, the DNA selective dye is Hoechst 33342. Another embodiment further comprises the step of orienting the entrained sperm cells or sperm nuclei. In another embodiment, the step of exposing the sperm cells or sperm nuclei to electromagnetic radiation comprises exposing the sperm cells or sperm nuclei to a laser beam with modified beam profile. In these embodiments, the sperm cells or sperm nuclei may comprise sperm cells or sperm nuclei from a first and a second mammalian species—in such embodiments, the first subpopulation of sperm cells or sperm nuclei comprises 25% to 75% of the sperm cells or sperm nuclei from the first mammalian species. In a further embodiment, determining the presence of a DNA aberration comprises detecting more than two peaks or modes, or a peak to valley ratio of 80% or less, 70% or less, 60% or less, or 50% or less, on a histogram of fluorescence intensities or measureable differences in co-efficient of variation between modes in multivariate plots. Another embodiment of the invention comprises subjecting the entrained sperm cells or sperm nuclei to orienting hydrodynamic forces such as those imparted by an orienting nozzle.

Another embodiment of the invention comprises a method of analyzing a fluorescence emission characteristic in sperm cells or sperm nuclei, comprising staining the sperm cells or sperm nuclei with a DNA selective dye; entraining the stained sperm cells or sperm nuclei in sheath fluid; exposing the entrained sperm cells or sperm nuclei to electromagnetic radiation; determining a fluorescence emission characteristic of the exposed sperm cells or sperm nuclei; generating a first multivariate plot based on the fluorescence emission characteristic; providing a first gate on the first multivariate plot; generating a second multivariate plot based on the first gate; providing a first sort region on the second multivariate plot encompassing a first subpopulation of sperm cells or sperm nuclei and a second sort region on the second multivariate plot encompassing a second subpopulation of sperm cells or sperm nuclei, wherein the first sort region encompasses the center of the second multivariate plot. In a further embodiment, the first subpopulation of sperm cells or sperm nuclei comprises 25% to 75% of the sperm cell or sperm nuclei population. In a yet further embodiment, the second subpopulation of sperm cells or sperm nuclei excludes the first subpopulation of sperm cells or sperm nuclei. Another embodiment of the invention comprises subjecting the entrained sperm cells or sperm nuclei to orienting hydrodynamic forces such as those imparted by an orienting nozzle.

In a yet further embodiment, determining the presence of a DNA aberration comprises detecting a difference between a peak to valley ratio on a histogram of fluorescence intensities of sperm cells or sperm nuclei from the first mammalian species and a peak to valley ratio or co-efficient of variation in modes on a histogram of fluorescence intensities of sperm cells or sperm nuclei from the second mammalian species.

Another embodiment of the invention comprises a composition comprising sonicated sperm cells or sperm nuclei, a DNA selective dye, an aggregation reducing compound and a buffer. In a further embodiment, the aggregation reducing compound comprises egg yolk, iodixanol, lecithin, bovine serum albumin, gelatin, collagen or hydrolyzed collagen, arabinogalactan, or a chemically defined polyethylene or polypropylene glycol. In a yet further embodiment, the sonicated sperm cells or sperm nuclei comprise sperm cells or sperm nuclei from a first non-human mammalian species and sperm cells or sperm nuclei from a second non-human mammalian species. In a further embodiment, the DNA selective dye is Hoechst 33342.

In a further embodiment of the invention, any of the methods for determining the presence or absence of a DNA aberration disclosed herein can further comprise the step of culling the male from whom the sperm cells or sperm nuclei are obtained or derived based on the determined presence of a DNA aberration in the male. In another embodiment of the invention, any of the methods for determining the presence or absence of a DNA aberration disclosed herein can further comprise the step removing the male, from whom the sperm cells or sperm nuclei are obtained or derived, from a breeding program in a genetic nucleus or a herd based on the determined presence of a DNA aberration in the male. In another embodiment, the method further comprises karyotyping the male from whom the sperm cells or sperm nuclei are obtained or derived based on the determined presence of a DNA aberration in the male. In an additional embodiment, the method further comprises karyotyping the dam of the male from whom the sperm cells or sperm nuclei are obtained or derived based on the determined presence of a DNA aberration in the male. In an additional embodiment, the method further comprises culling the dam of the male from whom the sperm cells or sperm nuclei are obtained or derived based on the determined presence of a DNA aberration in the male or an abnormal karyotype in the dam. In a further embodiment, the method further comprises determining the presence or absence of a DNA aberration (using any of the methods disclosed herein) in, or karyotyping, the progeny of the dam of the male from whom the sperm cells or sperm nuclei are obtained or derived based on the determined presence of a DNA aberration in the male. In a further embodiment, the method further comprises culling progeny of the dam of the male from whom the sperm cells or sperm nuclei are obtained based on the determined presence of a DNA aberration or abnormal karyotype in the progeny or the determined presence of a DNA aberration in the male.

It should be understood that any of the embodiments disclosed herein for determining the presence or absence of a DNA aberration can be employed to detect the presence or absence of the aberration in either sperm cells or sperm nuclei (i.e., sperm with their tails and midpieces removed, by for example, sonication)—that is, either sperm or sperm nuclei can be analyzed with the methods of the invention.

The invention also encompasses an improved method for making sperm nuclei comprising combining a DNA-selective dye and an aggregation reducing compound with a sperm cell sample to create a sperm cell mixture; and sonicating the sperm cell mixture to create stained sperm nuclei.

Another embodiment of the invention comprises a composition comprising unsorted sperm nuclei, an aggregation-reducing compound and a DNA selective dye, wherein the composition has been sonicated. In a further embodiment, the DNA selective dye is Hoechst 33342. In another embodiment, the sperm nuclei are derived from sperm cells from one male. In another embodiment, the composition has a temperature of 45° C. or greater. In a particular embodiment, the aggregation-reducing compound is egg yolk.

An additional embodiment of the invention comprises a method of processing sperm cells comprising providing an unsorted sperm cell sample; combining a DNA selective dye and an aggregation-reducing compound with the unsorted sperm cell sample to create a sperm cell mixture; and sonicating the sperm cell mixture to create stained sperm nuclei. In another embodiment, the DNA selective dye is Hoechst 33342. In a yet further embodiment, the unsorted sperm cell sample is obtained from one male. In another embodiment, the sperm cell mixture has a temperature of 45° C. or greater during the step of sonication. In a yet further embodiment, the aggregation-reducing compound is egg yolk. In an additional embodiment, the method is completed in 20 minutes or less.

An even further embodiment of the invention comprises a method of identifying a translocation in a male comprising providing a first subpopulation and a second subpopulation of sperm cells from a population of sperm cells from the male, wherein the first subpopulation comprises the 84^{th} percentile or higher of sperm cells in the population with respect to detected DNA quantity and the second subpopulation comprises the 16^{th} percentile or lower of sperm cells in the population with respect to detected DNA quantity; sequencing DNA from the first subpopulation and the second subpopulation; generating reads for the first subpopulation and reads for the second subpopulation; aligning the reads for the first subpopulation and the reads for the second subpopulation to a reference genome; averaging the base sequencing depth across non-overlapping windows for the first subpopulation and for the second subpopulation;
computing a scaled sequencing depth difference between the first subpopulation and the second subpopulation; and identifying a translocation in the male based on the scaled sequencing depth difference. In a more particular embodiment of the method, sequencing comprises short read sequencing. In a further embodiment, the step of sequencing is performed at a depth of 10× or lower. Another embodiment further comprises a step of mapping translocation breakpoints to single pairs of regions within a chromosome using the scaled sequencing depth difference. In yet another embodiment, the regions are 100 kbp in length or shorter. In an even more particular embodiment, the step of sequencing is performed at a depth of 10× or higher. In yet another embodiment, the detected DNA quantity is detected by a flow cytometer. In a specific embodiment, forward fluorescence intensity is used to detect DNA quantity.

Another embodiment of the invention comprises a method of processing sperm cells comprising staining a population of sperm cells with a DNA-selective dye; entraining the population in sheath fluid; exposing the entrained population to electromagnetic radiation to generate events for the population; gating a first subpopulation of the events, wherein each event within the first subpopulation corresponds to a sperm cell in the 84^{th} percentile or higher of sperm cells in the population with respect to detected DNA quantity; gating a second subpopulation of the events, wherein each event within the second subpopulation corresponds to a sperm cell in the 16^{th} percentile or lower of sperm cells in the population with respect to detected DNA quantity; separating the sperm cells corresponding to the gated first subpopulation from the population of sperm cells; separating the sperm cells corresponding to the gated second subpopulation from the population of sperm cells; and genotyping DNA from the separated sperm cells corresponding to the gated first subpopulation and DNA from the separated sperm cells corresponding to the gated second subpopulation.

The improved sperm nuclei of the invention can be used in any of the aforementioned methods of determining the presence or absence of a DNA aberration.

### BRIEF DESCRIPTION OF THE DRAWINGS

FIG. 1 shows examples of multivariate and univariate plots as well as gates used for logical gating of abnormal boar sperm nuclei (i.e., comprising a DNA aberration).

FIG. 2 illustrates part of a flow cytometer used to analyze and then sort a sperm composition to form one or more subpopulations.

FIG. 3 illustrates a microfluidic chip used to analyze and then sort a sperm composition.

FIG. 4 illustrates a univariate plot in the form of a histogram generated by an analyzer or sorter.

FIGS. 5-11 are flow cytometer images showing exemplary histograms for the five different categories of stable chromosomal translocation found in boars, produced using the parse sorting method of the invention.

FIGS. 12-18 are flow cytometer images showing histograms for boar sperm nuclei analyzed using the parse sorting method of the invention.

FIGS. 19-22 are flow cytometer images showing histograms for boar sperm nuclei analyzed using the logical gating method of the invention.

FIGS. 23-33 are flow cytometer images showing histograms for boar sperm nuclei analyzed using the parse sorting method of the invention.

FIG. 34 shows flow cytometer images showing histograms for bull sperm nuclei analyzed using the parse sorting method of the invention.

FIG. 35 shows a screenshot from a flow cytometer showing multivariate plots and a univariate plot of forward fluorescence intensities of a stained sperm cell sample indicating the presence of a DNA aberration (Category IV translocation) in a boar.

FIG. 36 shows a screenshot from a flow cytometer showing multivariate plots and a univariate plot of forward fluorescence intensities of a stained sperm cell sample indicating the presence of a DNA aberration (Category III translocation) in a boar.

FIG. 37 shows a screenshot from a flow cytometer showing multivariate plots and a univariate plot of forward fluorescence intensities of a stained sperm cell sample indicating the presence of a DNA aberration (Category IV translocation) in a boar.

FIG. 38 shows a flow cytometer image showing a histogram for a subpopulation from the center sort region indicating the presence of a DNA aberration in a bull.

FIG. 39 shows a flow cytometer image showing a histogram for a subpopulation from the flanking sort region indicating the presence of a DNA aberration in a bull.

FIG. 40 is a screenshot of a flow cytometer calibrated with normal sperm nuclei.

FIG. 41 is a screenshot of a flow cytometer analyzing sperm nuclei obtained from a boar with a mos t(7-9) chromosomal translocation.

FIG. 42 is a screenshot of a flow cytometer analyzing sperm nuclei obtained from a boar with a mos t(7-9) chromosomal translocation in which an additional amount of egg yolk was added to the sperm nuclei to improve orientation and resolution.

FIG. 43 is a screenshot of a flow cytometer analyzing sperm nuclei obtained from a boar with an rcp (3;6)_2 chromosomal translocation.

FIG. 44 is a screenshot of a flow cytometer analyzing sperm nuclei obtained from a boar with an rcp_1-7 chromosomal translocation.

FIG. 45 is a screenshot of a flow cytometer analyzing sperm nuclei obtained from a boar with an rcp (2q13,15q24) chromosomal translocation.

FIGS. 46 and 47 are screenshots of a flow cytometer analyzing sperm nuclei obtained from two boars without chromosomal translocations, respectively.

FIGS. 48A to 48D are graphical representations of florescence density patterns generated by sperm cell samples comprising different types of chromosomal translocations.

FIGS. 49A to 49D are univariate plots of forward fluorescence values generated by sperm cell samples from four bulls with chromosomal translocations, respectively.

FIG. 50A to 50D show G-banding karyotypes of four bulls with chromosomal translocations, respectively.

FIGS. 51A to 51D show the chromosomes involved in each translocation, determined by calculating the scaled sequencing depth difference between the upper and lower tail samples from four bulls, respectively.

FIGS. 52A to 52D show DNA breakpoints involved in chromosomal translocations present in four bulls, respectively.

### DETAILED DESCRIPTION OF THE INVENTION

One embodiment of the invention encompasses a method that uses a low-cost and readily available biomaterial in sperm, facilitates the rapid analysis of said sperm (or derived sperm nuclei) in a matter of minutes (in contrast to days or weeks,) and depending on the level of precision and accuracy of the equipment and operator, can generate statistically significant proof and quantification of changes in total DNA that may be as low as 0.1%. Parallelization of the invention in sample handling and total per-sample analysis time of less than one hour can facilitate the analysis of about 10 samples or more per day and relatedly as many as 2500 samples per year at costs per sample that may be as low as 20% of karyotype analysis costs. The invention is also capable of yielding a more sensitive measurement of the size of DNA aberrations than is typical with karyotyping. Using the invention, identity mapping to show identical genotypes (related carrier animals) is facilitated, and the study of the way such aberrations arise and inherit is also facilitated. It should be understood that the invention in any of its embodiments can be used to determine the presence or absence of a DNA aberration using either sperm or sperm nuclei.

Although the invention may substitute or replace higher cost and time consuming karyotyping, it is also complementary, as the measured feature is distribution of total DNA in different subpopulations without specification of which chromosomes are affected. In some embodiments, the invention can be performed using state-of-the-art equipment used in the sex-selection of mammals in industrial scale and can therefore be introduced to existing users of sex-selection technology with limited new capital investment.

Since in some embodiments, the invention can use frozen sperm and in further embodiments be micro-scaled to use as few as 50 million sperm, it can be performed using one or more cryopreserved semen straws, even from animals that are not living. In this way, even though in some embodiments the invention may be concentrated at locations that specialize in sex-selection in general, the shipment and contract analysis of samples will be possible.

Since the invention uses flow cytometry, future advances in miniaturization, automation and sensitivity of sperm analysis by flow cytometry will facilitate future reduction in cost of using the invention and parallelization of analysis, if needed. As used herein, the term “flow cytometry” also encompasses microfluidics, and the term “flow cytometers” encompasses both flow cytometers and microfluidic devices generally. Certain embodiments of the invention may further comprise the use of kits, where ready-to-use reagents and consumable materials can allow the dissemination of the method to large numbers of different locations with same quality of analysis.

In certain embodiments, the invention does not require the use of computational analysis of histogramatic data produced in flow cytometry (as exemplified in flow cytometry standard [“FCS”] files), but since such data can easily be produced during the analysis, the precision with which data can be analyzed can be improved by using a large number of already existing software packages for analyzing flow cytometry data. In other embodiments, computational analysis of the data, including histogramatic data, produced by the invention may be utilized.

It is contemplated that the invention can be applied to many hundreds of thousands or millions of breeding sires (e.g., boars and bulls) and can readily become a standard and essential method in the screening of breeding value and reproductive performance of livestock or other animals, including humans. Additionally, although methods for artificial chromosomes (synthetic autosomes) are still emerging, cattle and swine geneticists are developing and utilizing safe and effective methods of artificial transgenesis. In certain embodiments, the analysis of stability and integrity of heritability of large DNA transgenes that increase the total DNA content may be analyzed or studied by the invention, and it is contemplated that the use of artificial transgenes may be facilitated by sorting methods similar to the sex-selection methods used today.

Generally, in one aspect of the invention, a fresh or frozen ejaculate of sperm is the raw material to be processed. A DNA selective dye or stain (including, but not limited to, Hoechst 33342) is combined in a liquid media with appropriate pH, modifying buffers and aggregation reducing chemicals. The sperm mixture is then optionally sonicated to simultaneously provide heat that accelerates the rate of stain penetration and saturation and to disrupt sperm cell structure to remove tails and midpieces, thereby creating stained sperm nuclei.

One embodiment of the invention comprises entraining the stained sperm or sperm nuclei in sheath fluid and exposing the entrained sperm or sperm nuclei to electromagnetic radiation. In connection with individual events associated with the exposed sperm cells or sperm nuclei, the invention further comprises determining a forward fluorescence characteristic, which in certain embodiments correlates to the quantity of DNA in a sperm cell or a sperm nuclei and a side fluorescence characteristic, which in certain embodiments correlates to the orientation of a sperm cell or sperm nuclei. Next, the invention contemplates gating the individual events based on the forward fluorescence characteristic and side fluorescence characteristic with one or more criterion, e.g., oriented sperm or sperm nuclei, or DNA content. In certain embodiments of the invention, one can determine the presence or absence of a DNA aberration from the gated individual events. “Chromosomal aberration” or “DNA aberration” as used herein encompasses a chromosomal translocation, a transgene, or any other state in which the DNA content of sperm from an individual varies from cell to cell other than variation attributable to sex chromosomes. For example, a univariate plot can be generated based on forward fluorescence intensity of the gated individual events. Typically, in normal mammalian sperm or sperm nuclei, the univariate plot would show two distinct peaks (each peak corresponding to X-bearing or Y-bearing sperm). In an individual with a chromosomal or DNA aberration, however, the univariate plot may show more than two peaks. Additionally, in certain embodiments of the invention, a peak to valley ratio is calculated for the peaks shown on the univariate plot, with a peak to value ratio of 50% or less indicating the presence of a chromosomal aberration, for example. In related embodiments, comparison of co-efficient of variation between two or more peaks or modes using mathematical analysis (Gaussian or the like) from histogramatic data is used.

In a further embodiment of the invention, the sperm cells or sperm nuclei are analyzed using a “logical gating” method using a flow cytometer or microfluidic device. This embodiment comprises gating a subpopulation of oriented sperm or sperm nuclei on a multivariate plot with a first gate and then generating a second multivariate plot based on the first gate. The method further comprises creating a first “center gate” on the second multivariate plot that selects a percentage of sperm cells or sperm nuclei distributed near and around the mean of population distribution (two or more “peaks”), and a second “flanking gate” on the second multivariate plot that selects for the remaining sperm cells or sperm nuclei outside of the center gate or a substantial portion thereof. A univariate plot can be generated based on forward fluorescence intensity for each of the center gate and the flanking gate. Normal sperm cell or nuclei samples that comprise two peaks (normal X and normal Y) result in center and flanked populations that show the same two peaks on each of the univariate plots with similar peak to valley ratios, typically greater than 50%. In contrast, abnormal sperm cell or nuclei samples that contain smaller or larger autosomes or sets of autosomes will result in differences between the number of peaks that appear in the center and flanked populations or peak to valley ratios of 50% or less, for example.

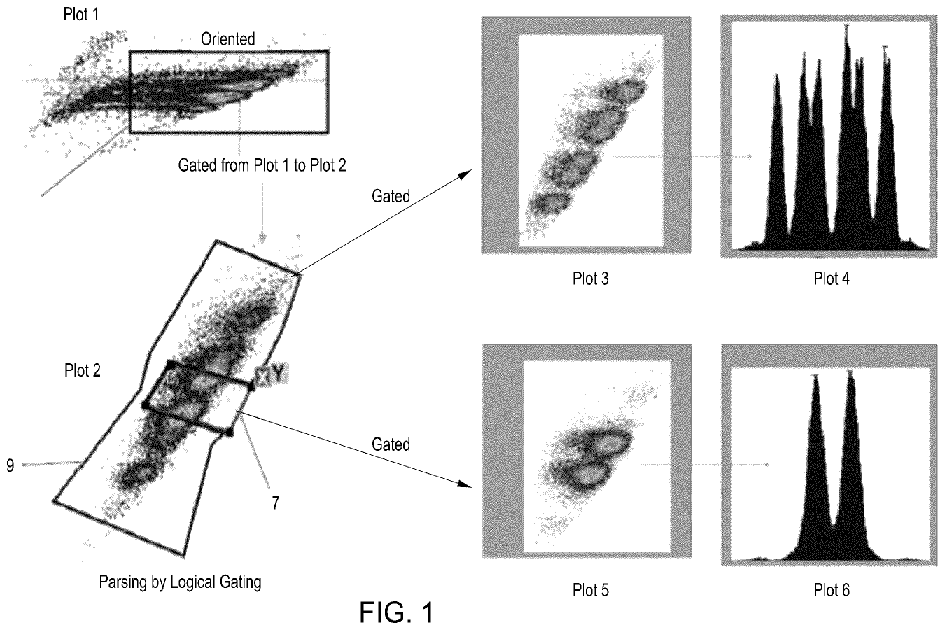

FIG. 1 shows examples of multivariate and univariate plots as well as gates used for logical gating of abnormal boar sperm nuclei (i.e., comprising a DNA aberration). Plot 1 represents a multivariate plot showing forward fluorescence along the y-axis and side fluorescence along the x-axis. A gate 5 has been placed around events that are oriented well enough to capture optimal resolution. Each dot represents an event detected by the flow cytometer. Events outside of gate 5 are low resolution or unresolvable. The events within gate 5 are then plotted on plot 2, which shows forward fluorescence along the y axis and an integral of forward fluorescence along the x-axis. Essentially, plot 2 depicts the gated events in plot 1 rotated approximately 90° towards the viewer. In plot 2, a center gate 7 and a flanking gate 9 are placed. The events within flanking gate 9 are then plotted on plot 3, which shows forward fluorescence intensity along the y axis and an integral of forward fluorescence intensity along the x-axis, and plot 4, which is a histogram showing the forward fluorescence intensities of events gated by the flanking gate 9. The events within center gate 7 are plotted on plot 5, which shows forward fluorescence intensity along the y axis and an integration of forward fluorescence intensity along the x-axis, and plot 6, which is a histogram showing the forward fluorescence intensities of events gated by the center gate 7. As can be seen in FIG. 1, the histogram (plot 4) generated by events in the flanking gate 7 shows an abnormal number of modes, or peaks (normal being two modes, or peaks, in mammalian species) and corresponds to a boar that has a chromosomal translocation.

In another embodiment of the invention, sperm cells or sperm nuclei are actually “parse sorted” using a flow cytometer or microfluidic device, where “parse sorting” encompasses sorting a first “center sort region,” selecting for a percentage of sperm cells or sperm nuclei distributed near and around the mean of population distribution (two or more “peaks”), and a second “flanking sort region,” which typically selects for the remaining sperm cells or sperm nuclei outside of the center sort region. In some embodiments, more than two subpopulations may be sorted, for example, a center sort region and two separate flanking sort regions. The separate, parse sorted sperm cell or sperm nuclei subpopulations are then analyzed and compared. Normal sperm cell or nuclei samples that comprise two peaks (normal X and normal Y) result in center and flanking populations that show the same two peaks. Abnormal samples that contain smaller or larger autosomes or sets of autosomes will result in differences in the number and relative DNA content of peaks that appear in the center and flanking populations.

In a further embodiment of parse sorting, the separate populations that are sorted may include further dividing the flanking sort region to separate the high-total-DNA content sperm cells or sperm nuclei on one side of the flank from the low-total-DNA content sperm cells or sperm nuclei on the other side of the flank, while also separating a population from the center where average-total-DNA content sperm cells or sperm nuclei are selected.

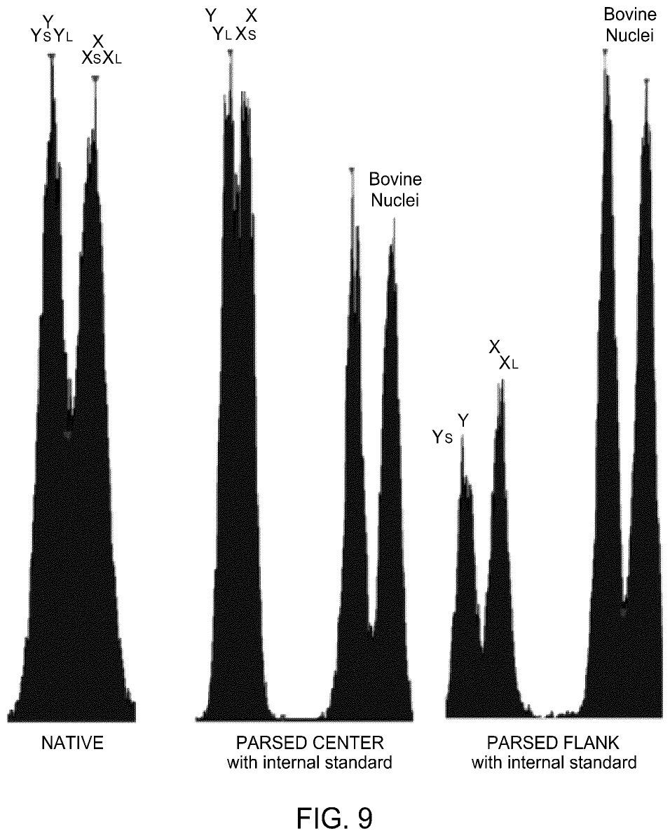

In a further embodiment of the invention, sperm cells or sperm nuclei of a second species may be added to the sperm cell or nuclei sample to provide an internal standard to establish a reference of known particle count, known total fluorescence intensity, known percent DNA differential between sex chromosomes, known peak to value ratio, or reference standard deviation of Gaussian distribution. In certain embodiments, a method for creating the internal standard comprises the addition of sperm or sperm nuclei of a known quantity from a secondary mammalian species to a staining mixture comprising a DNA selective dye with appropriate conditions of sonication, which can then be added as an internal standard to the sperm cell or nuclei sample of the first mammalian species that is to be analyzed for chromosomal aberrations. For example, FIG. 9 shows histograms for parsed center and parsed flank subpopulations of sperm nuclei from a boar that includes an internal standard comprising bovine sperm nuclei. The histogram for the parsed center subpopulation in FIG. 9 clearly indicates the presence of a DNA aberration in the boar's sperm nuclei since the peak to valley ratios for that subpopulation is significantly lower than the peak to valley ratio shown on the histogram of the internal standard.

In one embodiment, mathematical analysis of the Gaussian distributions of normal and abnormal peaks within single samples and across groups of samples can be used to make a differential determination of normal vs abnormal (i.e., the presence or absence of a DNA aberration) more sensitive and precise.

Generally, the invention is rapid. However, certain embodiments of the invention may take longer than other embodiments. For example, in some embodiments, sperm nuclei populations may be partially or fully purified away from tails and midpieces prior to staining. Additionally, in certain embodiments, staining may be performed at temperatures lower than in some embodiments of the invention. In some embodiments, the invention can use slower and more benign sample handling methods if maintaining the viability of sperm is required, such as foregoing sonication.

Some embodiments of the invention may specify a target temperature at which sonication is completed/terminated. The target temperature can be 5° C., 45° C., or 70° C., or can be in the range between 5-70° C., 30-70° C., or 55-65° C. In some embodiments of the invention the target temperature may be reached in a time between 1 minute and 5 minutes, with staining completed in that time.

In certain embodiments, the invention uses flow cytometry sorters or microfluidic devices that are identical to or materially similar to flow sorters used to sort live sperm for total DNA content in industrial sex selection. In certain embodiments of the invention, the flow cytometer or microfluidic device utilizes an event rate between 50 to 500 events per second, 5,000 to 30,000 events per second, or less than 30,000 events per second and sort rates of about 10-35% of the event rate. In certain embodiments, where sperm are not sorted before analysis, the analysis rates may be as low as 2-10% of the event rate.

For those embodiments encompassing parse sorting, the invention comprises sorting into two or more subpopulations, centered around the mean and flanked around the center, where the percentage of parsed center cells or nuclei (sort region percentage) is between 25-60% of total sperm cells or sperm nuclei, and the percentage of parsed flanked cells or nuclei is the complement/balance of cells or nuclei remaining and in some cases further dividing the flanking sort region to separate the high-total-DNA content sperm or sperm nuclei on one side of the flank from the low-total-DNA content sperm or sperm nuclei on the other side of the flank. Alternate embodiments that sort into more than two subpopulations or require the combinatorial blending of parse samples are contemplated for use in special situations.

Certain embodiments of the invention encompass extended sorting times to provide larger quantities of parse sorted samples that may then be analyzed by microstructural DNA methods such as genotyping or sequencing are anticipated.

In certain embodiments, the invention encompasses the separate analysis of the two or more subpopulations of parse sorted sperm or sperm nuclei (generally corresponding to center and flanking subpopulations). Any of the above methods can be applied to these separated subpopulations. For example, one may entrain the sperm cells or sperm nuclei, expose them to electromagnetic radiation, determine a forward fluorescence characteristic and a side fluorescence characteristic, gate the individual events based on the forward fluorescence characteristic and side fluorescence characteristic with one or more criterion and generate a univariate plot based on forward fluorescence intensity to determine the number of peaks or the peak to valley ratio. Alternatively, a parse sorted sperm cell or nuclei subpopulation can be reprocessed using the logical gating method or parse sorting method. Alternatively, the parse sorted sperm cells or sperm nuclei may be analyzed by micro-analysis of DNA such as gene chip analysis or next-generation sequencing (NGS).

In certain embodiments, it is contemplated that univariate plots (e.g., graphical histograms) are visually inspected for qualitative comparison of native and parse sorted samples. In other embodiments of the invention, it is contemplated that flow cytometry data from TRACE files or related FCS files is mathematically analyzed for quantitative comparison of native and parse sorted samples. Although the value of mathematical analysis of flow cytometry data from TRACE files or related FCS files may facilitate superior comparative quantification of and identity of related samples, embodiments of the invention that forego mathematical data analysis (e.g., using only the human eye) may be more sensitive than existing karyotype methods.

### Sperm Collection

It is contemplated that intact viable bovine, porcine, equine, ovine, cervine, murine or other mammalian sperm, may be collected for use with the invention. Various methods of collection of viable sperm cells are known and include, for example, the gloved-hand method, use of an artificial vagina, and electro-ejaculation. As an example, a bovine sperm sample, typically containing about 0.5 to about 10 billion sperm per milliliter, may be collected directly from the source mammal, or from more than one source mammal of the same species, into a vessel containing an extender to form a sperm cell composition. An extender may optionally comprise one or more antioxidants, which may be present as constituents of the extender prior to contacting with the sperm, or which may be added to the sperm cell composition, each antioxidant in the concentration range of 0.01 mg/ml to 5 mg/ml.

An aggregation reducing compound, or anti-aggregation compound, may also be added to the sperm cell composition to prevent aggregation of sperm. Examples of aggregation reducing compounds suitable for use in the invention include but are not limited to egg yolk, iodixanol, lecithin, bovine serum albumin, gelatin, collagen or hydrolyzed collagen, macromolecules such as arabinogalactan, and chemically defined polyethylene or polypropylene glycols.

### Staining Sperm Cells for Use in the Invention

A process of staining sperm for flow cytometric analysis typically comprises the formation of a staining solution containing sperm cells and a dye, sometimes referred to as a label. A media or extender may be contacted with sperm cells to form a sperm composition, and then the sperm composition contacted with a DNA selective dye to form a staining solution. Alternatively, a DNA selective dye may be added to a media or an extender to form a staining solution, with sperm subsequently added to the staining solution.

The sperm to be stained may be a neat semen (i.e., raw ejaculate), or alternatively, a sperm-containing semen derivative obtained by centrifugation or the use of other means to separate semen into fractions.

The pH of the staining solution may be maintained at any of a range of pHs; typically this will be in the range of about 5.0 to about 9.0, or in the range of 5.5 to 7.8. The staining solution may be maintained at a slightly acid pH, i.e., from about 5.0 to about 7.0. Typically, the pH is from about 6.0 to about 7.0; from about 6.0 to about 6.5; about 6.2, about 6.5; about 6.6; about 6.7; about 6.8; about 6.9; or about 7.0. Alternatively, the staining solution may be maintained at a slightly basic pH, i.e., from about 7.0 to about 9.0. Typically, the pH is about 7.0 to about 8.0; about 7.0 to about 7.5; about 7.0; about 7.1; about 7.2; about 7.3; about 7.35; about 7.4; or about 7.5.

The staining solution may be formed by using one or more UV or visible light excitable, DNA selective dyes as previously described in U.S. Pat. No. 5,135,759 and WO 02/41906, the contents of each of which are hereby incorporated herein by reference. Exemplary UV light excitable, selective dyes include Hoechst 33342 and Hoechst 33258.

The concentration of the DNA selective or of any other type of dye in the staining solution is a function of a range of variables which include the permeability of the cells to the selected dye, the temperature of the staining solution, the amount of time allowed for staining to occur, the concentration of sperm, and the degree of enrichment desired in the subsequent sorting or enrichment step. In general, the dye concentration is preferably sufficient to achieve the desired degree of staining in a reasonably short period of time. For example, the concentration of Hoechst 33342 in the staining solution will generally be between about 0.1 μM and about 1.0M; from about 0.1 μM to about 1000 μM; from about 100 μM to about 500 μM; from about 200 μM to about 500 μM; or from about 300 μM to about 450 μM. Accordingly, under one set of staining conditions, the concentration of Hoechst 33342 is about 350 μM. Under another set of staining conditions, the concentration of Hoechst 33342 is about 400 μM. Under still another set of staining conditions the concentration is about 450 μM. In some embodiments the concentration of Hoechst 33342 is between about 250 and about 500 picomoles of Hoechst per 1 million sperm.

Once formed, the staining solution may be maintained at any of a range of temperatures; typically, this will be within a range of about 4° C. to about 50° C., but can also be frozen (i.e., cryopreserved). For example, the staining solution may be maintained at a relatively low temperature, i.e., a temperature of about 4° C. to about 30° C.; in this embodiment, the temperature is about 20° C. to about 30° C.; from about 25° C. to about 30° C.; or about 28° C. Alternatively, the staining solution may be maintained within an intermediate temperature range, i.e., a temperature of about 30° C. to about 39° C.; in this embodiment, the temperature is at about 34° C. to about 39° C.; about 35° C.; or about 37° C. In addition, the staining solution may be maintained within a relatively high temperature range, i.e., a temperature of about 40° C. to about 50° C.; in this embodiment, the temperature is from about 41° C. to about 49° C.; from about 41° C. to about 45° C.; from about 41° C. to about 43° C.; or about 41° C. Selection of a preferred temperature generally depends upon a range of variables, including for example, the permeability of the cells to the dye(s) being used, the concentration of the dye(s) in the staining solution, the amount of time the cells will be maintained in the staining solution, and the degree of enrichment desired in the sorting or enrichment step.

Uptake of dye by the sperm in the staining solution is allowed to continue for a period of time sufficient to obtain the desired degree of DNA staining. That period is typically a period sufficient for the dye to bind to the DNA of the sperm such that X and Y chromosome-bearing sperm can be distinguished from one another based upon the differing and measurable fluorescence intensity between the two. Generally, this will be no more than about 24 hours; no more than about 30 hours; no more than about 10 hours; no more than about 2 hours; no more than about 90 minutes; no more than about 60 minutes; or from about 5 minutes to about 60 minutes. In a particular embodiment, the period is about 30 minutes or about 55 minutes. In another embodiment, the period is less than 5 minutes, less than 4 minutes, less than 3 minutes, about 2 minutes, or less than 2 minutes.

### Creating Stained Sperm Nuclei for Use in the Invention

One aspect of the invention comprises a staining media for making stained sperm nuclei. The stained sperm nuclei can in turn be used in the methods disclosed herein for detecting the presence or absence of a chromosomal aberration instead of using intact sperm cells.

In one embodiment, the staining media comprises a buffer, a DNA-selective dye and an aggregation-reducing compound. Any suitable buffer in the art, such as TRIS citrate, sodium citrate, sodium bicarbonate, HEPES, TRIS, TEST, MOPS, KMT, TALP, and combinations thereof, can be used.

Any DNA selective dye known in the art can be used, including but not limited to Hoechst 33342. In other embodiments, the staining media may be formed by using one or more UV or visible light excitable, DNA-selective dyes as previously described in U.S. Pat. No. 5,135,759 and WO 02/41906, the contents of each of which are hereby incorporated by reference. Exemplary UV light excitable, selective dyes include Hoechst 33342 and Hoechst 33258.

Additionally, an aggregation-reducing compound may be added to prevent aggregation of sperm cells or sperm nuclei, as well as aggregation of midpieces and tails. Examples of aggregation-reducing compounds suitable for use in the invention include but are not limited to egg yolk, iodixanol, lecithin, bovine serum albumin, gelatin, collagen or hydrolyzed collagen, macromolecules such as arabinogalactan, and chemically defined polyethylene or polypropylene glycols. In a particular embodiment of the invention, the staining media can comprise 0.4% or more egg yolk. In another embodiment, the staining media can comprise between 1-30%, 1-20%, 1-15%, 1-10%, 1-5%, 1-3%, 1-2%, or 0.2-1%, egg yolk.

The staining media, or its separate components, can then be combined with a sperm cell sample to create a sperm cell mixture. The sperm cell mixture is then sonicated in order to remove midpieces and tails from the sperm heads to create sperm nuclei, and to facilitate staining of the DNA within the sperm nuclei. In a particular embodiment of the invention, the sperm cell mixture is sonicated at a sufficient amplitude, frequency or duration to raise the temperature of the sperm cell mixture to more than 30, 40, 50, 60 or 70° C. in order to facilitate staining. In a particular embodiment, the temperature of the sperm cell mixture is raised to more than 50, 60 or 70° C. during sonication. In another embodiment, the temperature of the sperm cell mixture is raised to at least approximately 60° C. during sonication. In other embodiments, the target temperature during sonication can be 45° C., or 70° C., or can be in the range between 30-70° C., or 55-65° C. In another embodiment, sonication can be carried out on the sperm cell mixture for a particular duration to facilitate staining of sperm nuclei DNA. In one embodiment, the sperm cell mixture can be sonicated for greater than 1, 2, 3, 4 or 5 minutes. In another embodiment, the sperm cell mixture can be sonicated for a total of 1-5 minutes or in an even more particular embodiment, approximately 2-3 minutes. In a further embodiment of the invention, once the sperm cell mixture is sonicated, the tails and midpieces are removed from the mixture via any known method know in the art, including but not limited to filtration or centrifugation. Any suitable sonicator can be used to make the sperm nuclei. In a particular embodiment, a sonicator with a 20 mhz frequency can be used to make the sperm nuclei, such as Fisher Scientific Model FB120. In a more particular embodiment, the sonicator is set to an amplitude of 70%. Finally, one can check whether sonication was successful by examining the sonicated sperm cell mixture by microscope—the sonicated sperm cell mixture should substantially comprise sperm heads, with midpieces and tails removed and should be substantially free of intact sperm cells.

In a particular embodiment, the staining media is made by combining 98.0 ml of TRIS-based media, comprising 2% egg yolk, with 2.0 ml of Hoechst 33342 (8.1 mM of Hoechst 33342) to yield a final concentration of Hoechst 33342 of 160 μM in the staining media. 1.5 ml of this staining media is then combined with a sperm cell sample of 400 million sperm (extended or raw ejaculate) to create a sperm cell mixture. The sperm cell mixture is then sonicated to for about 2 minutes or until it reaches a temperature of about 60° C. This sonication step acts to remove the midpieces and tails from the sperm cells to create sperm nuclei (i.e., sperm heads devoid of midpieces and tails) and to facilitate entry and binding of the DNA-selective dye (in this case, Hoechst 33342) to the DNA within the sperm nuclei.

### Use of an Internal Standard With the Invention

In some embodiments, it is contemplated that an internal standard is mixed with the stained sperm cells or stained sperm nuclei before analysis or mixed with each of the parsed sorted subpopulations prior to analysis. The internal standard may be created from sperm of a different species from the parse sorted subpopulation or the sperm of interest that is to be analyzed. The internal standard can comprise sperm nuclei, in which case, the internal standard can be made in accordance with the above disclosure regarding making stained sperm nuclei.

The internal standard is combined with a sperm cell or nuclei sample or a parse sorted sperm or nuclei subpopulation prior to flow cytometric analysis. One aspect of the invention involves determining the presence or the absence of a DNA aberration based on a comparison of histograms of fluorescence intensity of the native (i.e., unsorted) sperm cells or sperm nuclei, or parse sorted subpopulations, with the internal standard. Generally, when the histograms of the native or parse sorted subpopulations have a peak to valley ratio that is less than the peak to valley ratio of the internal standard, a DNA aberration is present. Generally, the Gaussian distribution of modes in the internal standard DNA from the sperm cells or sperm nuclei of the second mammalian species and the Gaussian distribution of modes in the normal sample from DNA of the reference species represent a combination of standards which can then be compared to the Gaussian distribution of unknown samples to identify a DNA aberration.

In one particular embodiment, the internal standard is created using a staining media made by combining 49.0 ml of TRIS-based media comprising 20% egg yolk with 432 μl of Hoechst 33342 stain. 1.66 ml of sperm or sperm nuclei (at a concentration of 200 million/ml) are then added to the staining media. This mixture can then be sonicated to create stained sperm nuclei.

### Detecting or Determining the Presence or Absence of DNA Aberrations

One aspect of the invention comprises analyzing stained sperm cells or sperm nuclei via flow cytometry in order to detect or determine the presence or absence of DNA aberrations, such as chromosomal translocations. As noted above, in certain embodiments of the invention, sperm cells or sperm nuclei analyzed by flow cytometry are also optionally sorted based on falling within a center or flanking sort region in the context of the parse sorting method for detection of DNA aberrations. Commonly used and well known sperm cell analysis and sorting methods via flow cytometry are exemplified by and described in U.S. Pat. Nos. 5,135,759, 5,985,216, 6,071,689, 6,149,867, and 6,263,745; International Patent Publications WO 99/33956 and WO 01/37655; and U.S. patent application Ser. No. 10/812,351 (corresponding International Patent Publication WO 2004/088283), the content of each of which is hereby incorporated herein by reference.

In certain embodiments of the invention, analysis of sperm cells may be accomplished using any process or device known in the art for cell analysis including but not limited to use of a flow cytometer (including the use of a microfluidic chip), and optionally encompasses techniques for physically separating sperm from each other, as with droplet sorting and fluid switching sorting, and techniques in which sperm bearing an undesired characteristics are killed, immobilized, or otherwise rendered infertile, such as by use of laser ablation/photo-damage techniques. Based on the fluorescence emitted by a DNA selective dye upon exposure to a light source such as a high intensity laser beam, a flow cytometer (including a microfluidic device) is able to measure or quantify the amount of DNA present in each cell stained with the DNA selective dye.

A sperm cell or nuclei sample to by analyzed via a flow cytometer (including a microfluidic device) is contained in a sample fluid. A sheath fluid is generally used in a flow cytometer or microfluidic device to hydrodynamically focus, entrain or orient sperm or nuclei in the sample fluid. Generally, the sheath fluid is introduced into a nozzle of a flow cytometer or into a microfluidic device using pressurized gas or by a syringe pump. The pressurized gas is often high quality compressed air. In certain embodiments of the invention, a stream containing sperm cells or nuclei to be analyzed may be comprised of a sample fluid and a sheath fluid, or a sample fluid alone. Optionally, the sample fluid or sheath fluid may also contain an additive, such as, one or more antioxidants, an antibiotic or a growth factor, as discussed above with respect to sperm sample collection. Each of these additives may be added to either fluid in accordance therewith.

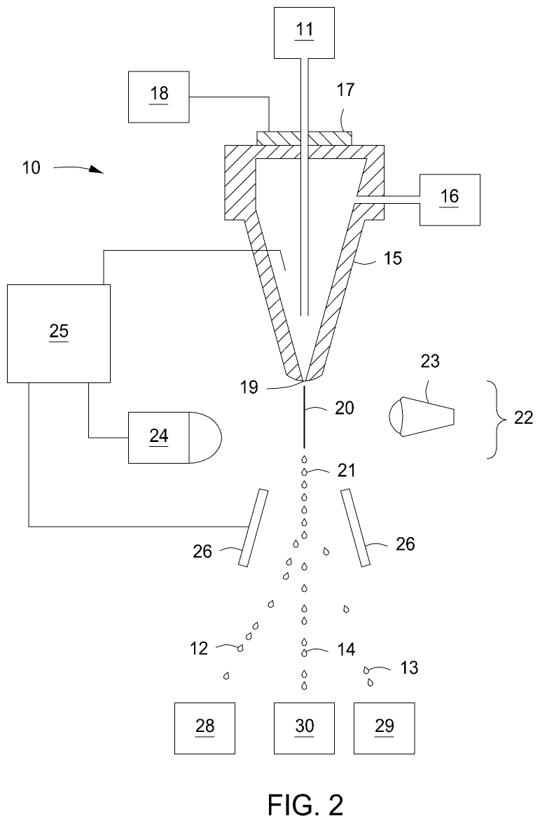

FIG. 2 illustrates, in schematic form, part of a flow cytometer used to analyze and then sort a sperm or nuclei composition to form one or more subpopulations, the flow cytometer being generally referenced as 10. The flow cytometer 10 of FIG. 2 can be programmed by an operator to generate two charged droplet streams, one containing cells or nuclei within a center sort region charged positively 12, for example, one containing cells or nuclei within a flanking sort region charged negatively 13 for example, while an uncharged undeflected stream of indeterminate or undesired cells or nuclei 14 simply goes to waste, each stream collected in receptacles 28, 29, and 30, respectively.

Initially, a stream of sperm cells or nuclei under pressure, is deposited into the nozzle 15 from the sperm cell or nuclei source 11 in a manner such that they are able to be coaxially surrounded by a sheath fluid supplied to the nozzle 15 under pressure from a sheath fluid source 16. An oscillator 17 which may be present can be very precisely controlled via an oscillator control mechanism 18, creating pressure waves within the nozzle 15 which are transmitted to the coaxially surrounded sperm cell or nuclei stream as it leaves the nozzle orifice 19. As a result, the exiting coaxially surrounded sperm cell or nuclei stream 20 could eventually and regularly form droplets 21.

The charging of the respective droplet streams is made possible by the cell sensing system 22 which includes a laser 23 which illuminates the nozzle exiting stream 20, and the light emission of the fluorescing stream is detected by a sensor 24. The information received by the sensor 24 is fed to a sorter discrimination system 25 which very rapidly makes the decision as to whether to charge a forming droplet and if so which charge to provide the forming drop and then charges the droplet 21 accordingly.

A characteristic of X chromosome bearing sperm cells or nuclei is that they absorb more fluorochrome dye than Y chromosome bearing sperm cells or nuclei because of the presence of more DNA, and as such, the amount of light emitted by the laser excited absorbed dye in the X chromosome bearing sperm cell or nuclei differs from that of the Y chromosome bearing sperm cells or nuclei. One of the difficulties in accurate quantification of sperm DNA using fluorescence is the geometry of the sperm head, which is shaped like a paddle in most species. Generally, the intensity of fluorescence is lowest when the flat face of the sperm is oriented toward a fluorescence detector. This flat orientation actually results in the most accurate measure of DNA content within a cell and thus, in sex sorting applications, the best discrimination between X and Y chromosome bearing sperm subpopulations. It is therefore desirable that only properly oriented sperm or sperm nuclei are considered in determining the presence or absence of a chromosomal aberrations. There are many techniques known in the art used to orient sperm using various forces generated by the flow cytometer and/or microfluidic device, all of which are contemplated for use with the invention. One way in which orientation can be accomplished in a flow cytometer is by using an orienting nozzle such as described in U.S. Pat. No. 6,357,307, which is hereby incorporated by reference in its entirety. In one embodiment of the invention, two detectors are used for detecting fluorescence emitted by sperm cells or nuclei. One of the detectors is oriented at 0° relative to the laser beam or other source of electromagnetic radiation and is used to measure forward fluorescence, which corresponds to cell DNA content. The second detector is oriented 90° relative to the laser beam and is used to measure side fluorescence, which corresponds to the orientation of the sperm cell or nuclei. Since the fluorescence signal is highest for sperm cells or nuclei oriented with their paddle edge toward the side fluorescence detector, only the sperm cells or nuclei that emit peak fluorescence to the side fluorescence detector are considered oriented by the flow cytometer.

The charged or uncharged droplet streams pass between a pair of electrostatically charged plates 26, which cause them to be deflected either one way or the other or not at all depending on their charge into respective collection vessels 28 and 29 to form a subpopulation of sperm cells or sperm nuclei that fell within the center sort region and a subpopulation of cells or nuclei that fell within the flanking sort region, respectively. The uncharged non-deflected sub-population stream containing undesired or indeterminate cells or nuclei go to the waste container 30.

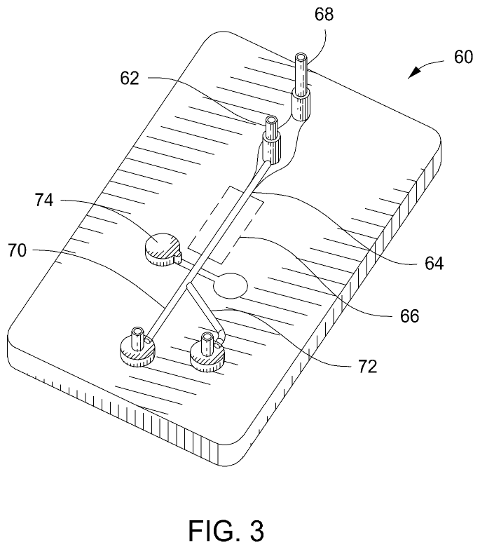

Turning now to FIG. 3, an alternative particle sorting instrument or flow cytometer is partially illustrated in the form of a microfluidic chip (60). The microfluidic chip (60) may include a sample inlet (62) for introducing sample containing particles or cells into a fluid chamber (64) and through an inspection zone (66). Sample introduced through the sample inlet (62) may be insulated from interior channel walls and/or hydrodynamically focused with a sheath fluid introduced through a sheath inlet (68). Sample may be interrogated at the inspection zone (66) with an electromagnetic radiation source (not shown), such as a laser, arc lamp, or other source of electromagnetic electricity. Resulting emitted or reflected light may be detected by a sensor (not shown) and analyzed with an analyzer (not shown). Each of the sheath pressure, sample pressure, sheath flow rate, and sample flow rate in the microfluidic chip may be manipulated in a manner similar to a jet-in-air flow cytometer, by either automatic adjustments performed by the execution of written instructions in the analyzer or by manual adjustments performed by an operator.

In certain embodiments of the invention, once inspected, particles or cells in the fluid chamber (64) may be mechanically diverted from a first flow path (70) to a second flow path (72) with a separator (74), for altering fluid pressure or diverting fluid flow. The particles or cells may also be permitted to continue flowing along the first flow path (70) for collection. The illustrated separator (74) comprises a membrane which, when depressed, may divert particles into the second flow path (72). Other mechanical or electro-mechanical switching devices such as transducers and switches may also be used to divert particle flow.

Flow cytometry data analysis is based on the principle of gating. Typically, gates and regions are created around populations of cells with common characteristics. In the context of the invention, these characteristics can include forward fluorescence and side fluorescence.

Generally, the first step in gating when flow cytometrically analyzing sperm cell or sperm nuclei to determine the presence or absence of a DNA aberration is distinguishing populations of sperm cells or sperm nuclei based on their forward and side fluorescence properties. As noted above, forward and side fluorescence provide an estimate of the DNA content of the cells or nuclei and their orientation, respectively. Unoriented sperm cells or nuclei will generate events having a lower level of side fluorescence, as noted above, and are not resolvable or are low resolution. These events can be removed by gating on the population of interest only (i.e., oriented sperm cells or nuclei).

Gates can be applied to density plots or contour maps to exclude certain populations (e.g. unoriented sperm cell or nuclei) or to positively select populations for further analysis, processing or examination. Using analytical software, measurements and statistics can be obtained for various parameters in addition to the number of cells or nuclei and percentage of cells or nuclei within a gate. This can include such measurements as median and mean fluorescence intensity.

Generally, two-parameter density plots (i.e., bivariate plots) display two measurement parameters, one on the x-axis and one on the y-axis and the events as a density (or dot) plot. The parameters can include forward florescence intensity, side fluorescence intensity and an integral of forward florescence intensity.

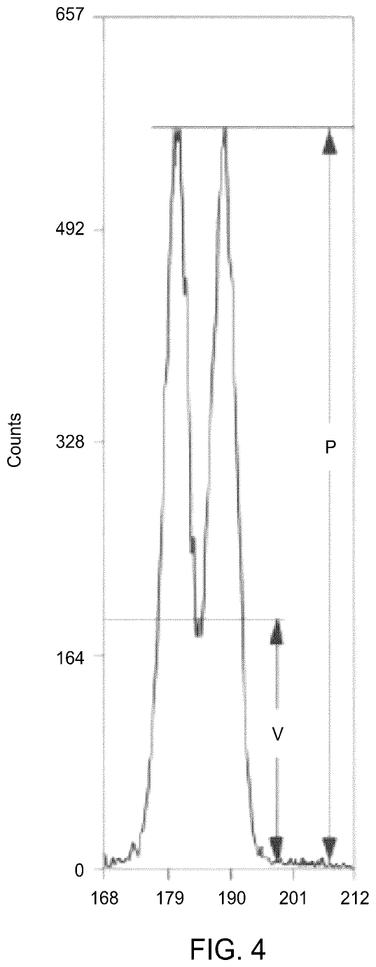

FIG. 4 illustrates a univariate plot in the form of a histogram that may be produced by the analyzer (36) and generated into a graphical presentation for an operator. The data illustrated in FIG. 4 may represent the number of occurrences of peak signal intensities from the side or forward fluoresce within a certain period. In the case of sperm cells or sperm nuclei, X chromosome bearing sperm cells or nuclei and Y chromosome bearing sperm cells or nuclei tend to have peak intensities that vary by between 2 and 5%, depending on the species, and this difference is reflected in the bimodal distribution of peak intensities. Because X chromosome bearing sperm cells or nuclei and Y chromosome bearing sperm cells or nuclei tend to have differing fluorescence values, each of the peaks represents either X chromosome bearing sperm cells or nuclei or Y chromosome bearing sperm cells or nuclei. FIG. 4 further illustrates the concept of the peak to valley ratio, which is derived from a relative intensity measurement at the lowest point between the two groups, the valley, which may be considered a value V, and a second relative intensity measurement at the peak or peaks of the histogram at P. In reference to FIG. 4 and for purposes of the invention, “peak to valley ratio” may be calculated by subtracting the value for V from the value for P (the average of the intensity measurements of the two largest peaks) and dividing the difference by the value for P:

(P−V)/P.

In addition to detecting or determining the presence or absence of a DNA aberration using a univariate plot of forward fluorescence intensities (e.g., FIG. 4) as described above, the invention also encompasses a method for detecting or determining the presence or absence of a DNA aberration, by analyzing or visualizing, or based on, forward fluorescence vs side fluorescence intensities (e.g., plot 1 in FIG. 1) or forward fluorescence vs an integral of forward fluorescence (e.g., plot 2 in FIG. 1) of sperm cells or sperm nuclei. As can be seen in plot 1 and plot 2 of FIG. 1, in addition to the two sub-populations associated with normal X and Y bearing chromosomes, at least two additional distinct sub-populations can be seen on either side of the normal X and Y subpopulations (i.e., above and below), indicating the presence of a DNA aberration.

A further embodiment of the invention comprises selecting a male based on the presence or absence of a DNA aberration in the male.

A specific embodiment comprises selecting a male in whom a DNA aberration is detected or determined for culling, and then culling the male. An alternative embodiment comprises removing a male in whom a DNA aberration is detected from a pool of prospective parents in a breeding program or within a genetic nucleus or removing the male from a genetic nucleus. Additionally, upon detecting the presence of a DNA aberration in a male, a specific embodiment of the invention further comprises conducting one or more additional tests on cells (including gametes and somatic cells) from the male, including but not limited to karyotyping, DNA sequencing, genotyping, proteomic, transcriptomic and metabolomic analyses, and histological examination. Additionally, a further embodiment comprises testing cells from the sire, mother, siblings or half-siblings of a male in whom a DNA aberration is detected, using any of the above-referenced tests, as well as using the flow cytometric methods of the invention. If a DNA aberration is also detected in the sire, mother, sibling or half-sibling of a male, one embodiment of the invention comprises culling the sire, mother, sibling or half-sibling of the male or making mating decisions for those individuals within a breeding plan based on detection of the presence of absence of a DNA aberration. Any known method in the art for detecting, measuring, quantifying or assaying molecules or other substances within or extracted from cells may be used with the invention, including but not limited to molecular hybridization, immunohistochemistry, real time quantitative PCR, quantitative reverse transcription PCR, blotting, nucleotide sequencing, protein sequencing, nuclear magnetic resonance spectroscopy, mass spectroscopy, liquid chromatography, gas chromatography and electrophoresis. In a specific embodiment, transcriptomes obtained from cells may be profiled using a microarray.

Another embodiment of the invention comprises selecting or identifying a male in whom the absence of a DNA aberration is detected or determined as a prospective or actual parent in a breeding program or within a genetic nucleus, breeding or mating the male (via natural mating or artificial insemination or other assisted reproductive technologies) and producing offspring from the male. A specific embodiment comprises selecting or identifying a male in whom the absence of a DNA aberration is detected as a donor of gametes for use in artificial insemination (including but not limited to IVF and intra-cytoplasmic sperm injection) and then producing or obtaining gametes from the male. Another embodiment of the invention comprises selecting or identifying a male in whom the absence of a DNA aberration has been detected for the production of sperm cells to be subjected to sperm sorting, including “sex-sorting” (which includes techniques for skewing sex-chromosome ratios by ablating or killing sperm cells with the undesired sex chromosome, in addition to physically separating X- and Y-chromosome bearing sperm cells), obtaining sperm cells from the male and then sorting the sperm cells. An additional embodiment of the invention comprises selecting a male in whom the absence of a DNA aberration is detected for cloning and then cloning the male.

Another embodiment of the invention comprises identifying low fertility males (e.g., having fertility rates below the average fertility rate for the species) in a population and then subjecting sperm cells from those males to the methods of the invention for detecting DNA aberrations.

### Rapid Method to Detect or Determine the Presence or Absence of a DNA Aberration

One embodiment of the invention encompasses a rapid method for determining the presence or absence of a DNA aberration comprising creating stained sperm nuclei from sperm cells by sonication in a composition comprising a DNA-selective dye and an aggregation-reducing compound, flow cytometrically determining forward and side fluorescence intensities generated by the stained sperm nuclei and determining the presence or absence of a DNA aberration based on the generated forward and side fluorescence intensities. The forward and side fluorescence intensities generated by sperm nuclei having a DNA aberration will typically form more than two subpopulations or clusters or, conversely, at least two subpopulations or clusters will be present but will be obscured or otherwise rendered indistinct from one another. This is in contrast to normal sperm cells or nuclei, in which the forward and side fluorescence intensities will form only two distinct subpopulations, consisting of normal X-chromosome bearing and normal Y-chromosome bearing sperm cells or nuclei. In an additional embodiment of the invention, the presence of a DNA aberration can be detected with the rapid method based on a histogram of forward fluorescence intensities. An animal with a chromosomal translocation will generate a histogram with more than two peaks or modes, or will generate a histogram with a peak-to-valley ratio of less than 90%.

Stained sperm cells or stained sperm nuclei may be prepared using the methods described hereinabove. Stained sperm nuclei in particular maybe generated in accordance with the above methods by sonicating sperm cells in a composition comprising a DNA-selective dye and an aggregation-reducing compound, such as egg yolk.

### Sequencing of Sorted Sperm Cells to Detect and Confirm Translocations

One embodiment of the invention comprises a method of detecting and/or confirming the presence of a DNA aberration, such as a chromosomal translocation, from genotype data obtained by sequencing certain subpopulations of sperm cells within a sperm cell sample. In a particular embodiment of the invention, a chromosomal translocation can be detected by first flow cytometrically separating certain subpopulations of sperm cells within a sperm cell sample and then sequencing those subpopulations in order to obtain genomic data. A chromosomal breakpoint can, in turn, be identified/detected from the genomic data, including the region of the breakpoint.

Each sperm cell (or each sperm nucleus) within a sample can fall into one of the following categories:
“Y”=Sperm cells or sperm nuclei that comprise a completely normal chromosome content (normal autosomes) and a Y chromosome. Sperm or sperm nuclei that comprise the short translocation (deleted) and long translocation (insert) in combination with other normal autosomes and a Y chromosome. NOTE: This designated mode contains two genotypes (two different types of chromosomal combinations) but the same amount of total DNA.
“X”=Sperm cells or sperm nuclei that comprise a completely normal chromosome content (normal autosomes) and an X chromosome. Sperm cells or sperm nuclei that comprise the short translocation (deleted) and long translocation (insert) in combination with other normal autosomes and an X chromosome. NOTE: This designated mode contains two genotypes (two different types of chromosomal combinations) but the same amount of total DNA.
“YS”=Sperm cells or sperm nuclei that contain the short translocation (deletion) with all other normal autosomes and a Y chromosome.
“XS”=Sperm cells or sperm nuclei that contain the short translocation (deletion) with all other normal autosomes and an X chromosome.
“YL”=Sperm cells or sperm nuclei that contain the long translocation (insert) with all other normal autosomes and a Y chromosome.
“XL”=Sperm cells or sperm nuclei that contain the long translocation (insert) with all other normal autosomes and an X chromosome.

One embodiment of the invention first comprises entraining sperm cells or sperm nuclei stained with a DNA selective dye in sheath fluid; exposing the entrained sperm cells or sperm nuclei to electromagnetic radiation; determining two or more fluorescence emission characteristics of individual events associated with the exposed sperm cells or sperm nuclei; and gating the individual events based on the two or more fluorescence emission characteristics with a criterion for further processing. A subpopulation comprising “XL” sperm cells (or sperm nuclei) can then be obtained by placing a first sort region on a univariate or bivariate plot of the gated individual events, in which the first sort region encompasses the gated individual events in the 84^{th} percentile or higher in terms of forward fluorescence intensity (i.e., DNA content). Additionally, a subpopulation comprising “YS” sperm cells (or sperm nuclei) can be obtained by placing a second sort region on the univariate or bivariate plot of the gated individual events, in which the second sort region encompasses the gated individual events in the 16^{th} percentile or lower in terms of forward fluorescence intensity (i.e., DNA content). These separated “upper” and “lower tail” subpopulations can then be subjected to DNA extraction and DNA sequencing.

In certain embodiments of the invention, a subpopulation comprising XL sperm cells (or sperm nuclei) can be obtained from a sperm cell sample by placing a sort region on a univariate or bivariate plot of gated individual events, in which the sort region encompasses only the gated individual events in the 84^{th} percentile or higher in terms of forward fluorescence intensity (i.e., DNA content). Likewise, a subpopulation comprising YS sperm cells (or sperm nuclei) can be obtained from a sperm cell sample by placing a sort region on the univariate or bivariate plot of the gated individual events, in which the sort region encompasses only the gated individual events in the 16^{th} percentile or lower in terms of forward fluorescence intensity (i.e., DNA content).

Next, DNA can be extracted from the subpopulations comprising XL or YS sperms cells using any suitable method for DNA extraction known in the art. By way of example only, the following DNA extraction and amplification procedure may be used in certain embodiments of the invention. One skilled in the art will know that variations on this method exist and that this method should not be construed to limit the functionality or scope of the current invention. This method is illustrative only.

1.5 ml tubes containing a cell suspension of cells from an individual are spun at ≥10000×g in a microcentrifuge for 45 seconds to pellet the cells. The suspension solution is pipetted off carefully so as to not remove the pelleted cells. Approximately 50 μl of suspension solution is left in each tube. The tubes are then vortexed for 10 seconds to resuspend the cell pellets. 300 μl of Tissue and Cell Lysis Solution (Epicentre; Madison Wisconsin; Catalog #MTC096H) containing 1 μl of Proteinase K (Epicentre; Madison Wisconsin; at 50 ug/μl; Catalog #MPRK092) is then added to each tube and mixed. The tubes are incubated at 65° C. for 30 minutes and vortexed at 15 minutes. The samples are cooled to 37° C. Afterwards 1 μl of 5 mg/μl RNase A (Epicentre; Madison Wisconsin; at 5 mg/ml; Catalog #MPRK092) is added to each sample and then mixed. The samples are then incubated at 37° C. for 30 minutes. The samples are then placed in a 4° C. cooler for 5 minutes. 175 μl of MPC Protein Precipitation Reagent (Epicentre; Madison Wisconsin; Catalog #MMP095H) is added to each sample, and the samples vortexed vigorously for 10-15 seconds. The samples are centrifuged in order to pellet debris for 8 minutes at ≥10000×g. The supernatant is transferred to a clean microcentrifuge tube. 600 μl of cold (−20° C.) isopropanol is added to the supernatant. Each tube is then inverted 30-40 times. The DNA is pelleted by centrifugation for 8 minutes in a microcentrifuge at ≥10000×g. The isopropanol is poured off without dislodging the DNA pellet. The pellet is rinsed once with 70% ethanol and then the ethanol is carefully poured off so as not to disturb the DNA pellet. The residual ethanol is removed with a pipet, and the DNA pellet is allowed to air dry in the microcentrifuge tube. Once dried, the DNA pellet is resuspended in 20 μl Tris-EDTA.

In certain embodiments of the invention, DNA from sperm cells or nuclei can be extracted using the Purelink Genomic Kit Cat #K1820-00 (Invitrogen). In further embodiments, once the DNA is extracted, it can be put through a whole genome amplification protocol using the Illustra Genomiphi V2 DNA amplification kit (GE Lifesciences), which uses the phi29 DNA polymerase to amplify the genome.

In certain embodiments of the invention, nucleic acid extracted from sperm cells or nuclei is then sequenced using any method known in the art, including, but not limited to, Sanger sequencing and high throughput sequencing, which includes next generation (short read) sequencing and third generation (long read) sequencing. In one embodiment of the invention, one read with short read sequencing comprises approximately 100 to 300 base pairs, and one read with long read sequencing comprises approximately 10,000 or more base pairs. Nonlimiting examples of sequencing methods for use in the invention include single-molecule real time sequencing, ion semiconductor sequencing, pyrosequencing, sequencing by synthesis, combinatorial probe anchor synthesis, sequencing by ligation, nanopore sequencing, massively parallel signature sequencing, polony sequencing, DNA nanoball sequencing, heliscope single molecule sequencing, and sequencing using droplet based microfluidics or digital microfluidics.

Alternatively, in some embodiments, extracted and/or amplified DNA from sperm cells or nuclei may be genotyped using genomic single nucleotide polymorphism (SNP) arrays or chips, which are readily available for various species of animals from companies such as Illumina and Affymetrix.

By sequencing subpopulations comprising XL and YS sperm cells or nuclei, reads are generated for these subpopulations. These reads are then aligned to a reference genome. Once this is completed, the base sequencing depth across non-overlapping windows is averaged for each of the XL and YS subpopulations. Next, a scaled sequencing depth difference can be computed between the XL and the YS subpopulations, which reveals the chromosome(s) involved in a translocation. In addition, the scaled difference in sequencing depth can be used to map the translocation breakpoint(s) to single pairs of candidate regions (i.e., genomic sequences comprising a translocation breakpoint) of 100 kbp or shorter or even to single base pair positions. Given that certain chromosomal translocations are heritable, a further embodiment of the invention comprises screening the progeny of a translocation carrier for the presence of the translocation. This is accomplished by genotyping or sequencing the DNA of the progeny (extracted for example from a somatic cell of the progeny) and identifying the genomic sequence(s) comprising the previously identified translocation breakpoint(s). In a particular embodiment, IVF embryos can be generated from sperm cells from a known translocation carrier. Cells from the embryos can then be harvested either in vitro, or in vivo after transfer into a recipient (as via amniocentesis). DNA can then be extracted from the harvested cells and sequenced or genotyped to determine if the embryo is a carrier of the translocation.

### Example 1

Frozen thawed sperm cell samples of seven different boars were centrifuged and then gently resuspended in TRIS A, an isotonic salt solution comprising a TRIS CITRATE buffer with FRUCTOSE and 20% v/v clarified Egg Yolk pH 6.80 and 300 mOsm. A 3-5 ml sample of suspended sperm cells (concentrations in the range of 200-300 million sperm per milliliter) was combined with Hoechst 333342 in ratio of 500-700 nanomoles of stain per 1 million sperm. The mixture was sonicated for 2-5 minutes until the temperature of the mixture reached 60° C., thereby creating stained sperm nuclei.

In the parse sorting method, one sort region is used to collect cells near the center (center sort region) while the other sort region is used to collect the remaining cells adjacent to the center sort region on both sides (flanking sort region). The combined percentages of both sort regions was typically about 90% of the cells. Generally, the percentage in the center sort region was somewhat less than the percentage in the flanking gate. The event rate during parse sorting of the native sample were typically in the range of 10,000-25,000 cells per second. The actual event rate chosen for each sample is determined by the operator and may change during the sorting.

For each of the seven boars exemplified here, the stained sperm nuclei samples were flow cytometrically sorted for the center sort region and flanking sort region into a collection tube. The sorted cells were centrifuged and decanted to remove most of the fluid. The cells were resuspended in approximately 300 microliters of the same buffer used for pre-sort staining and sonicating supplemented with about 100 nanomoles of Hoechst 33342 per million sperm and sonicated for 60 seconds before analysis. Since the purpose of the analysis is to have high quality resolution, and since slower event rates give higher quality resolution due to much smaller core stream diameters, event rates for analysis were typically 50-200 cells per second. The actual event rate chosen for each sample is determined by the operator and may change during the analysis.

Depending upon whether the boars were normal or had a DNA aberration, the number of peaks in the monovariate histogram were between 2 and 6 and the quality (peak to valley ratios) and peak clarity (co-efficient of variation) in the samples varied. Although the different “native samples” had different numbers of peaks, the distribution of peaks was typically symmetric around a central point. In normal boars showing only two peaks (X chromosome and Y chromosome bearing sperm population) the center was between those two peaks. In some boars with DNA aberrations there only appeared one poor quality peak and in some abnormal boars there appeared the typical X and Y peaks near the center as well as additional clearly defined peaks outside the two central peaks.

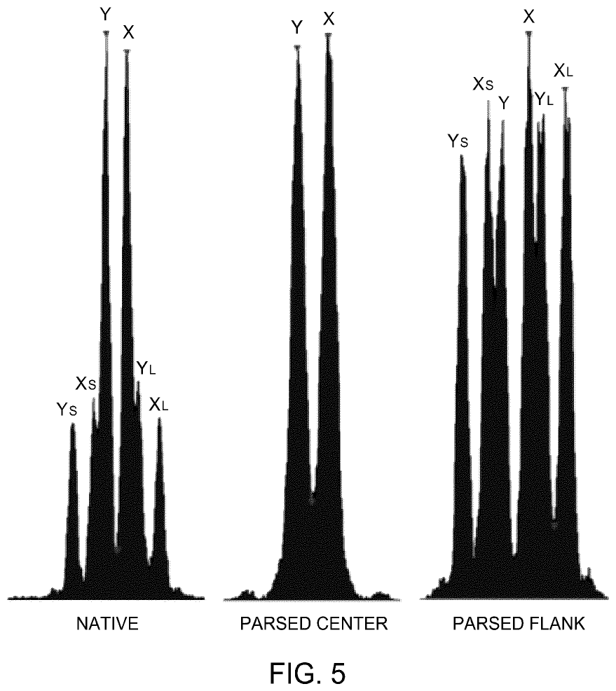

FIGS. 5 through 11 (each Figure corresponding to one of the seven boars, respectively) encompass 5 different categories of stable chromosomal translocation in found in boars, and the effect the size of translocated DNA in each of those categories has on the distribution of peaks (modes) in monovariate histograms of the sperm populations when the total DNA content is measured by the parse-sorted method. In each figure, the NATIVE histogram represents the data that was used for sorting (parse-sorted method). The PARSED CENTER histogram represents the data from sperm populations that were sorted by the CENTER SORT REGION and reanalyzed on the sorter. The PARSED FLANK histogram represents the data from sperm populations that were sorted by the FLANKING SORT REGION and reanalyzed on the sorter. The labels representing the 6 peaks (modes) are:
Y=Sperm cells or sperm nuclei that comprise a completely normal chromosome content (normal autosomes) and a Y chromosome. Sperm or sperm nuclei that comprise the short translocation (deleted) and long translocation (insert) in combination with other normal autosomes and a Y chromosome. NOTE: This designated mode contains two genotypes (two different types of chromosomal combinations) but the same amount of total DNA.
X=Sperm cells or sperm nuclei that comprise a completely normal chromosome content (normal autosomes) and an X chromosome. Sperm cells or sperm nuclei that comprise the short translocation (deleted) and long translocation (insert) in combination with other normal autosomes and an X chromosome. NOTE: This designated mode contains two genotypes (two different types of chromosomal combinations) but the same amount of total DNA.
YS=Sperm cells or sperm nuclei that contain the short translocation (deletion) with all other normal autosomes and a Y chromosome.
XS=Sperm cells or sperm nuclei that contain the short translocation (deletion) with all other normal autosomes and an X chromosome.
YL=Sperm cells or sperm nuclei that contain the long translocation (insert) with all other normal autosomes and a Y chromosome.
XL=Sperm cells or sperm nuclei that contain the long translocation (insert) with all other normal autosomes and an X chromosome.

Based on a convention comparing the amount of DNA in the stable translocation to the amount of DNA difference between X and Y chromosome, there are 4 categories of visible (measurable) aberrant DNA and the category of normal.

FIG. 5 depicts the parse-sorted analysis of a Boar with a Mos t(7-9) translocation that is typical of a Category I Translocation. In this case, since the number of base pairs in the translocated element is enough greater than the number of base pairs in the difference between an X chromosome and a Y chromosome, not only does the YS mode contain less DNA than the Y mode, the XS mode also contains less. Due to that, there are two modes visible below the Y mode. Conversely, there are also two modes larger than the X mode and even in the NATIVE histogram, it is possible to see 6 separate peaks. In the analysis of Category I PARSED CENTER and PARSED FLANK subpopulations that have been parse sorted and reanalyzed separately, the PARSED CENTER histogram is similar to a NORMAL XY distribution, but the PARSED FLANK histogram shows all 6 peaks very clearly. In this category, the aberrant DNA structure of the translocation can already be seen in the NATIVE histogram and is more clearly confirmed in the reanalyzed samples.

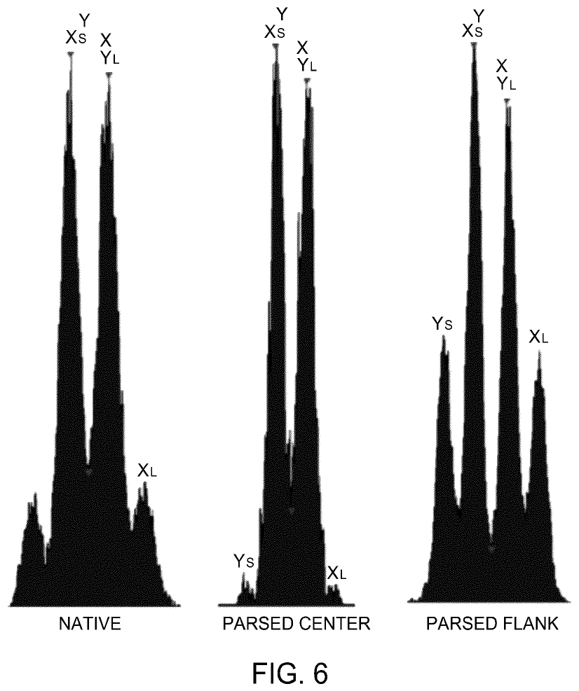

FIG. 6 depicts the parse-sorted analysis of a Boar with an RCP (3-6)2 translocation that is typical of a Category II Translocation. In this case, since the number of base pairs in the translocated element is about the same as the number of base pairs in the difference between an X chromosome and a Y chromosome, the XS mode tends to overlap well with the Y mode and the YL mode tends to overlap well with the X mode. In this case, both the NATIVE and the PARSED FLANK histograms show 4 peaks (modes). In this category, the aberrant DNA structure of the translocation can already be seen in the NATIVE histogram and is more clearly confirmed in the reanalyzed samples.

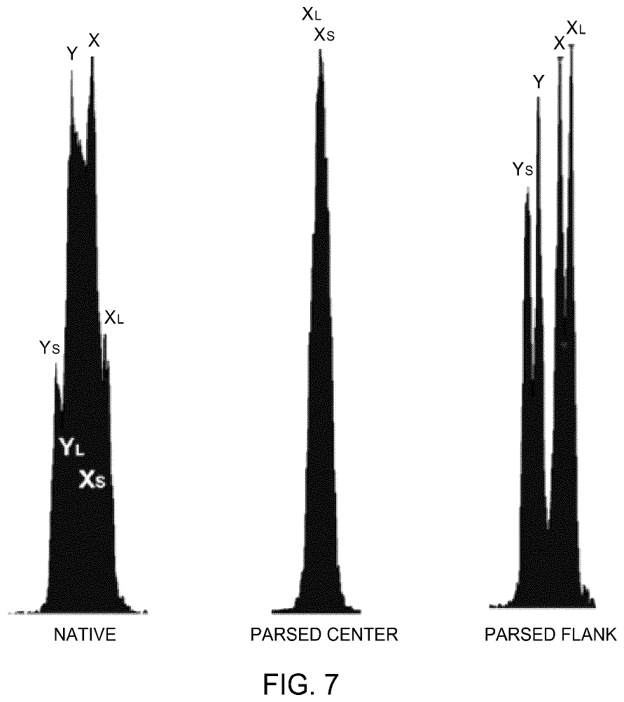

FIG. 7 depicts the parse-sorted analysis of a Boar with an RCP (2q13, 15q24) translocation that is typical of a Category III Translocation. In this case, since the number of base pairs in the translocated element is somewhat less than the number of base pairs in the difference between an X chromosome and a Y chromosome, the 4-6 peaks are poorly separated in the NATIVE histogram, but more clearly separated in the PARSED FLANK histogram. The PARSED CENTER histogram may still show two peaks (modes) that significantly overlap, or even show as one peak. A distinct feature of the Category III Translocation is that the PARSED FLANK histogram shows 4 peaks (modes).

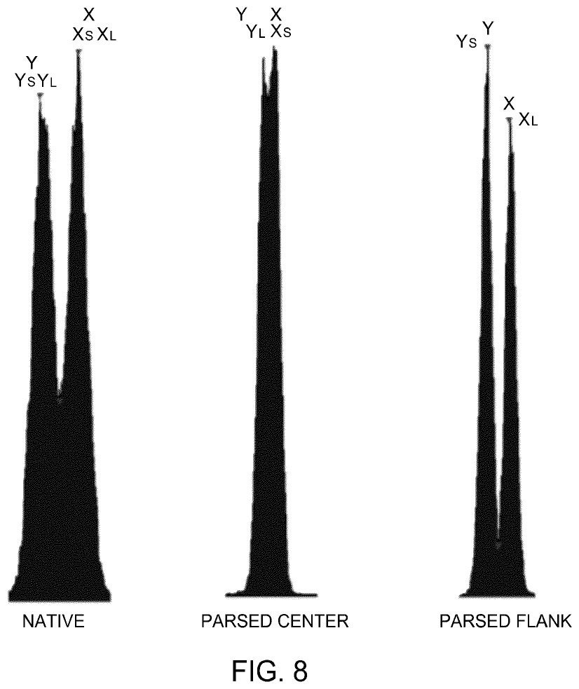

FIG. 8 depicts the parse-sorted analysis of a Boar with an RCP (5-12) translocation that is typical of a Category IV Translocation. In this case, since the number of base pairs in the translocated element is very small, the X modes (XS, X, XL) are all overlapping in the NATIVE histogram, while the Y modes (YS, Y, YL) are also overlapping in the NATIVE histogram and the NATIVE histogram looks similar to the NATIVE histogram of a NORMAL distribution (a boar with no large aberrant DNA). The Category IV Translocation still shows significant differences in the PARSED CENTER and PARSED FLANK analysis, but if the DNA element is very small, the only difference may be different CV (or PVR) between them.

FIG. 9 depicts the parse-sorted analysis of a boar with an RCP (5-12) translocation that is typical of a Category IV Translocation where the reanalysis of the PARSED CENTER and PARSED FLANK populations is done with normal sperm nuclei from cattle (bovine, Jersey breeds) included in the analysis. The bovine sperm nuclei contain more total DNA than the boar DNA, so the bovine sperm nuclei create a typical two-mode split seen in sex sorting but shifted to the right. Since both types of sperm nuclei are being analyzed together, the quality of the split (separation of modes) in the bovine nuclei is used to optimize the analysis and prove to the operator that the best possible separation has been created. That means that the best possible separation has also been created in the boar sperm nuclei test sample.

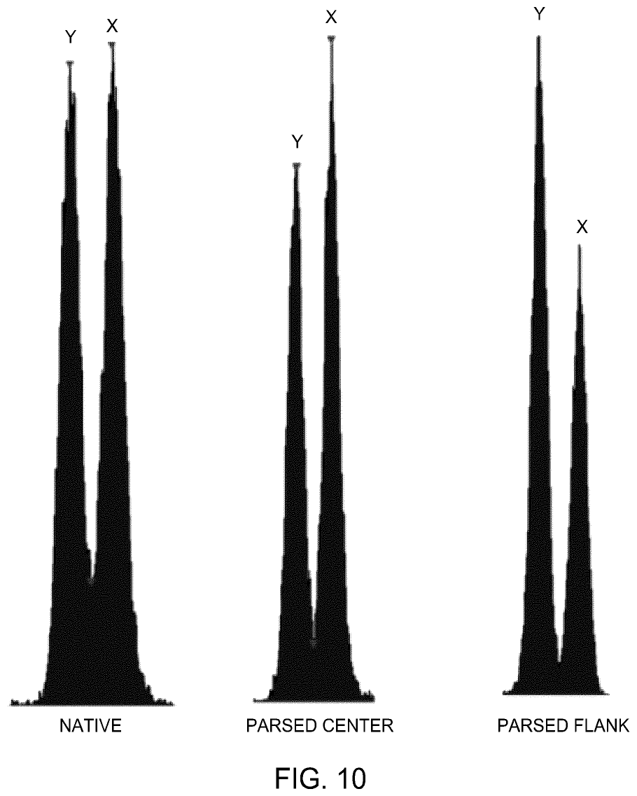

FIG. 10 depicts the parse-sorted analysis of a Boar with NORMAL DNA content. In this case, the designation of NORMAL has been created by the observation that the boar, when analyzed by Karyotype method, shows no recognizable abnormality in chromosome sizes. In this case, the NATIVE, PARSE CENTER and PARSE FLANK analysis all show 2 distinct peaks. The somewhat improved separations in the PARSE CENTER and PARSE FLANK are created because the reanalysis runs at a slow Event Rate (cell analysis rate) which typically gives better separation.

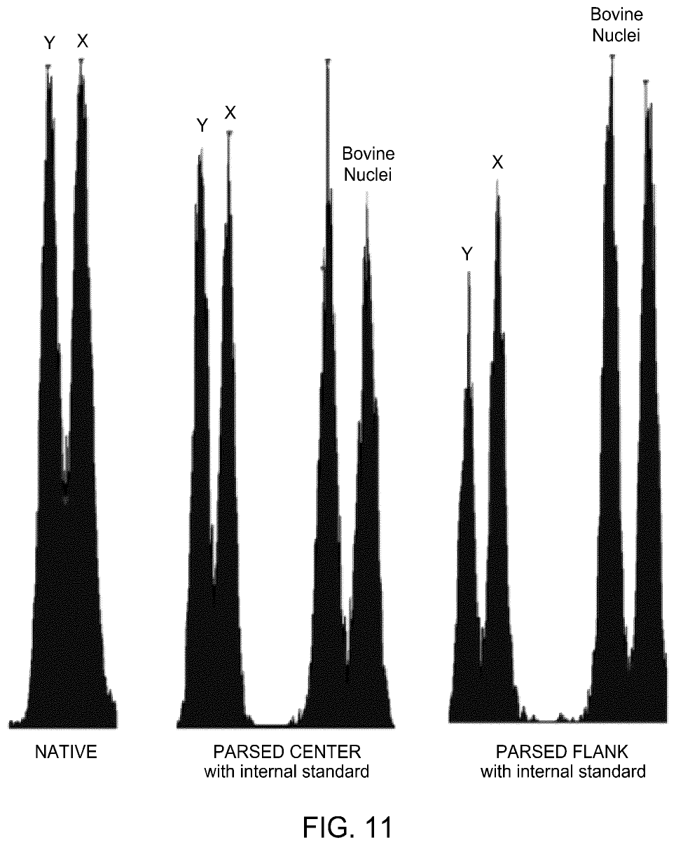

FIG. 11 depicts the parse-sorted analysis of a boar with NORMAL DNA content where the reanalysis includes bovine nuclei as described in FIG. 9. The parse-sorted method on NORMAL samples establishes a threshold of NORMAL vs false positive (falsely described as abnormal when there is no aberrant DNA content) vs false negative (falsely described as normal when there is an aberrant DNA content)

### Example 2

STAINING/SORTING MEDIA (SSM) used in Example 2—One liter comprises 23.00 grams tris(hydroxymethyl)aminomethane (TRIS BASE), 9.56 grams anhydrous fructose, 13.28 grams citric acid monohydrate, 200 mL chicken egg yolk, 100 milligrams Hoechst 33342, Trihydrochloride, Trihydrate and approximately 800 mL of deionized water with a final pH about 6.72.

SORT REANALYSIS MEDIA (SRM) used in Example 2—One liter comprises: 25.88 grams tris(hydroxymethyl)aminomethane, 10.75 grams anhydrous fructose, 14.94 grams citric acid monohydrate, 100 mL chicken egg yolk, 21.5 milligrams Hoechst 33342, Trihydrochloride, Trihydrate and approximately 900 ml of deionized water with a final pH about 6.72. If bovine nuclei are included as internal standard, 6.70 billion Bovine Sperm Nuclei (BSN) where BSN are azide stabilized purified sperm heads that have all midpieces and tail fragments removed and which have been pre-stained with Hoechst 33342.

Ejaculate preparation: Frozen ejaculates from 7 boars were thawed at 34° C. and each ejaculate then mixed well using a 5 ml pipette. 7 ml of each ejaculate was placed into a 15 ml conical tube. 7 ml of TrisWS300 was placed into each sample and vortexed. Samples were then centrifuged at 950 G for 5 minutes, and all supernatant was then decanted leaving the pellet undisturbed. 4 ml of TrisWS300 was added to each sample and the pellet was broken up using a pipette. Each sample was then mixed by vortexing. Cell concentrations were determined using NucleoCounter.

Sample preparation using SSM media: 615 μl of each of the prepared ejaculates was then placed in a 5 ml tube (assuming the sample concentration was 650 million sperm per ml). Using a P-1000 pipette, 400 million sperm cells from each sample were dispensed into a 5 ml sample tube. Using a P-1000 pipette, 1000 μl of SSM (prepared as above) was dispensed into the same tube. Each sample was then sonicated for 2 minutes, taking the samples to approximately 60° C. 500 ul of Tris A was then added to each sample and then each sample was sonicated again for ˜30 seconds.

Each of the 7 stained sperm nuclei samples was then parse sorted by placing 5.0 million sperm nuclei from each sample into a 5 ml catch tube and sorting them on a flow cytometer. A CENTER sort region and a FLANK sort region were placed on the bivariate plot of forward fluorescence peak vs forward fluorescence integrated. The sorted samples (2 for each bull) were then centrifuged at 950 G for 5 minutes, and all supernatant was decanted leaving the pellet undisturbed. 500 μl of SRM (as prepared above) was then added to each sample and the pellet was broken up with a sonicator for 1 minute. Each sorted sample was then analyzed on a flow cytometer at an event rate of between 40-100 events per second.

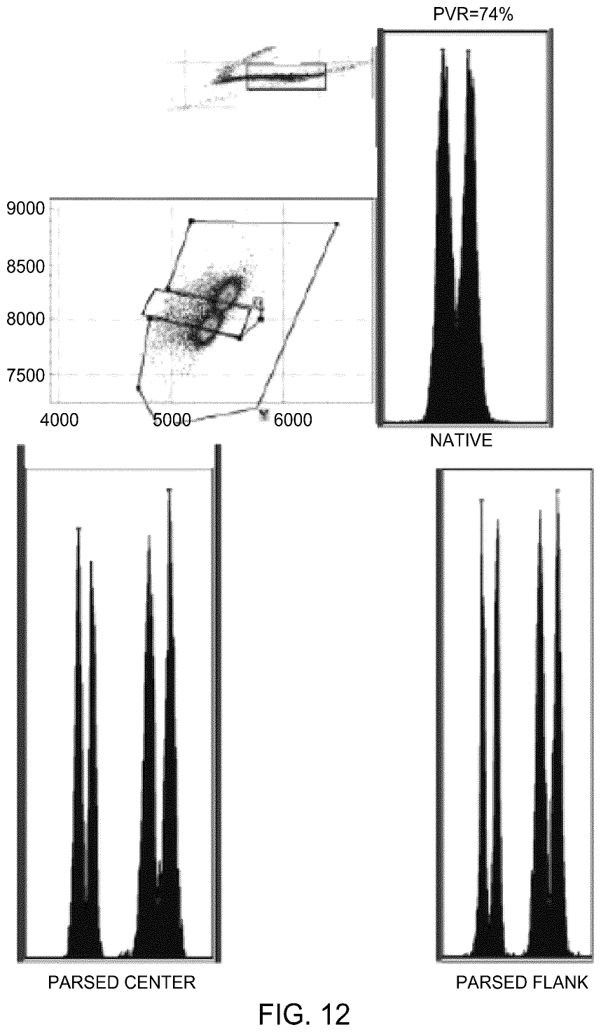

FIGS. 12 through 18 comprise the 7 examples of using the SSM staining media, sorting of SSM stained boar nuclei with the parse sort method, and reanalysis of the PARSED CENTER and PARSED FLANK populations using the SRM media, as indicated above. In all cases, the upper left side of the image shows two dot plots: the first shows an oriented gate applied to SSM stained sperm in a bivariate plot of forward fluorescence (FF) vs side fluorescence (SFF) and the second shows a set of CENTER and FLANK region polygons that define two separate sort regions on a bivariate plot of forward fluorescence (FF) vs forward fluorescence integrated (FFI, Area). The first monovariate histogram is the NATIVE histogram used for parsed sorting, the second monovariate histogram is the PARSED CENTER sorted sperm with bovine stained nuclei added and the third monovariate histogram is the PARSED FLANK sorted sperm with bovine stained nuclei added.

FIG. 12 depicts the parse-sorted analysis of a Boar with NORMAL DNA content where the reanalysis includes bovine stained nuclei as described above for FIG. 9.

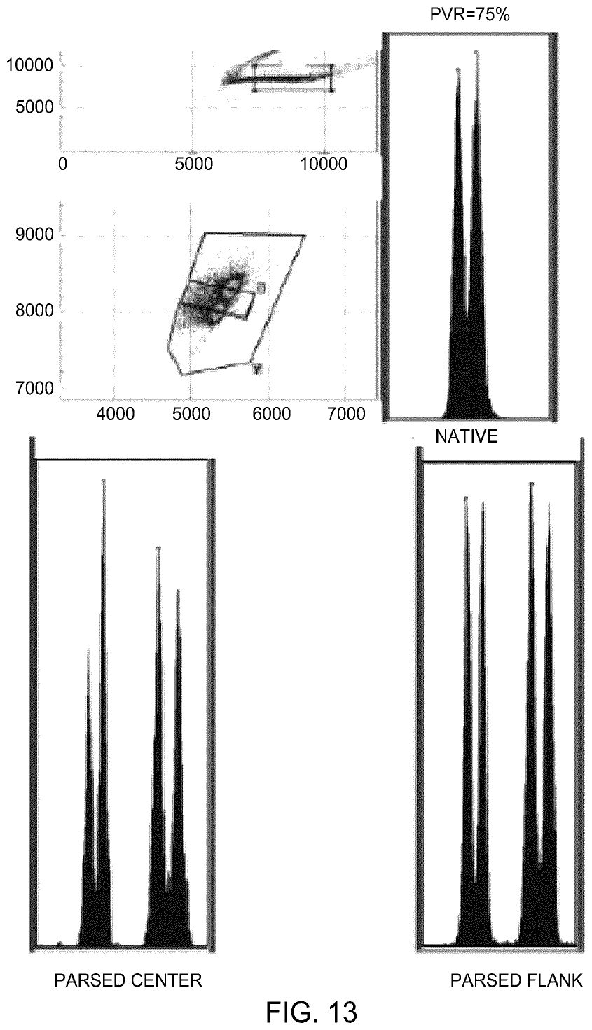

FIG. 13 depicts the parse-sorted analysis of a Boar with NORMAL DNA content where the reanalysis includes bovine stained nuclei as described above for FIG. 9.

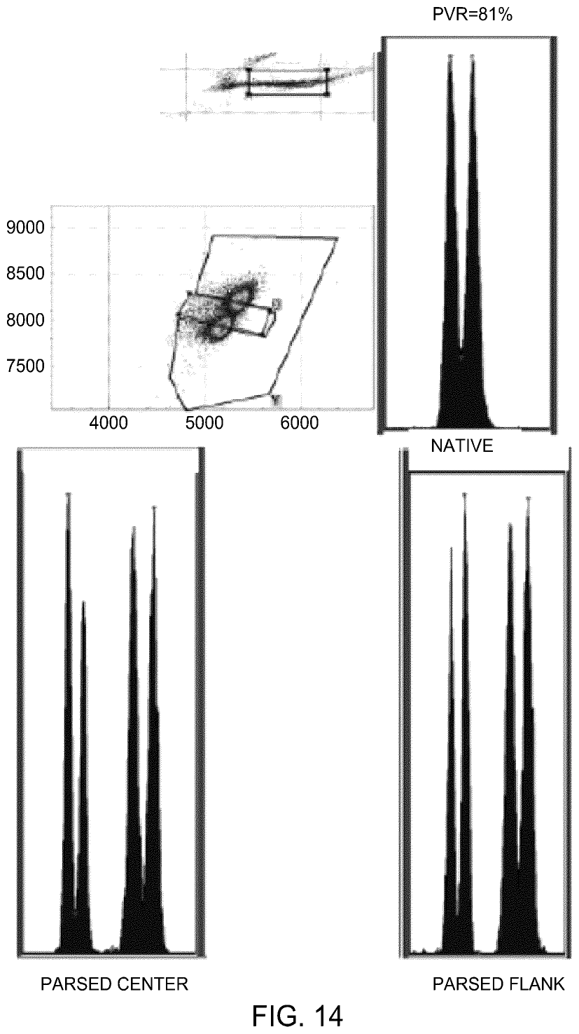

FIG. 14 depicts the parse-sorted analysis of a Boar with NORMAL DNA content where the reanalysis includes bovine stained nuclei as described above for FIG. 9.

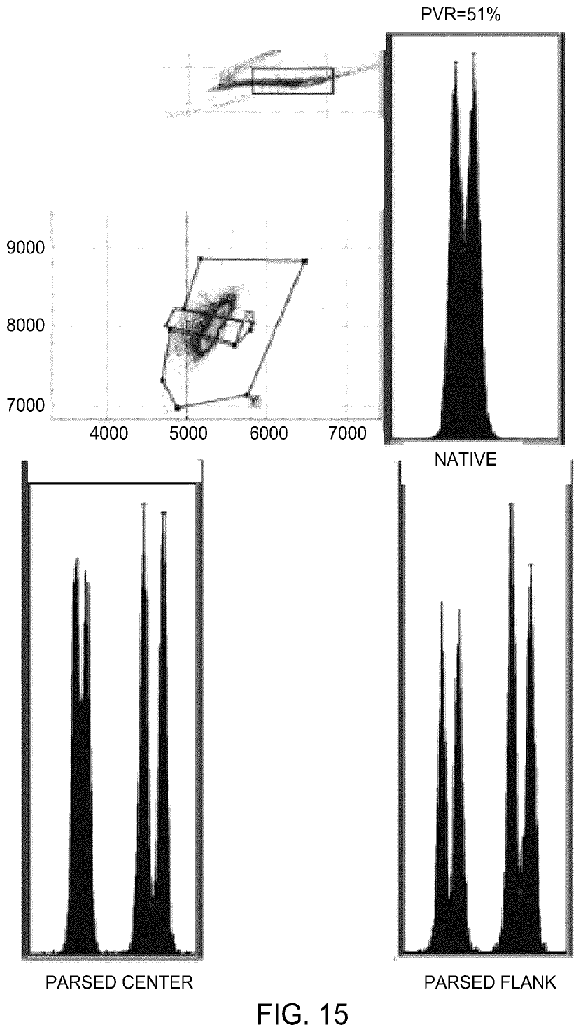

FIG. 15 depicts the parse-sorted analysis of a Boar with a Category IV Stable Reciprocal Translocation confirmed by Karyotype, with aberrant DNA content where the reanalysis includes bovine stained nuclei as described above for FIG. 9.

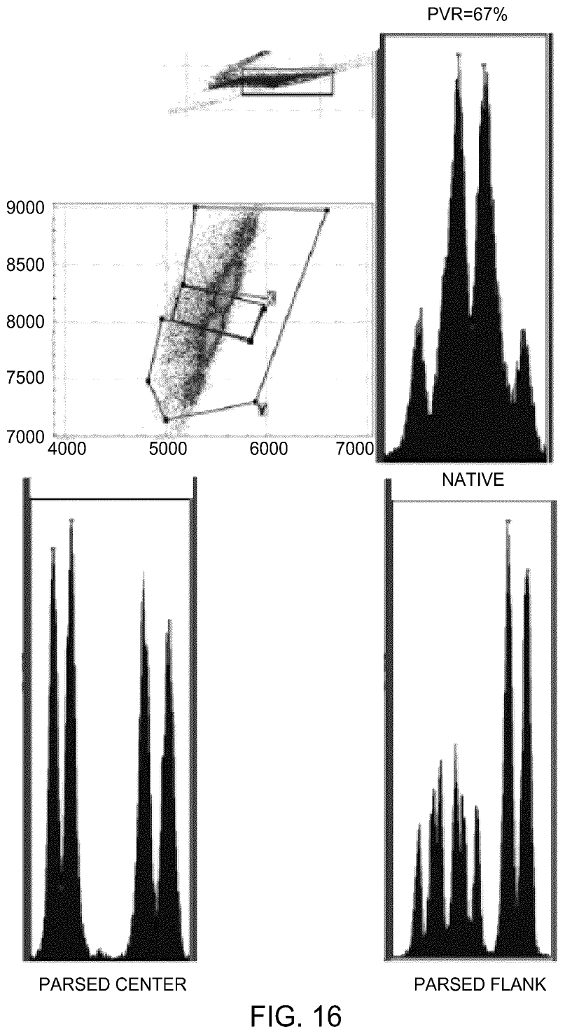

FIG. 16 depicts the parse-sorted analysis of a Boar with a Category I Stable Reciprocal Translocation confirmed by Karyotype, with aberrant DNA content where the reanalysis includes bovine stained nuclei as described above for FIG. 9.

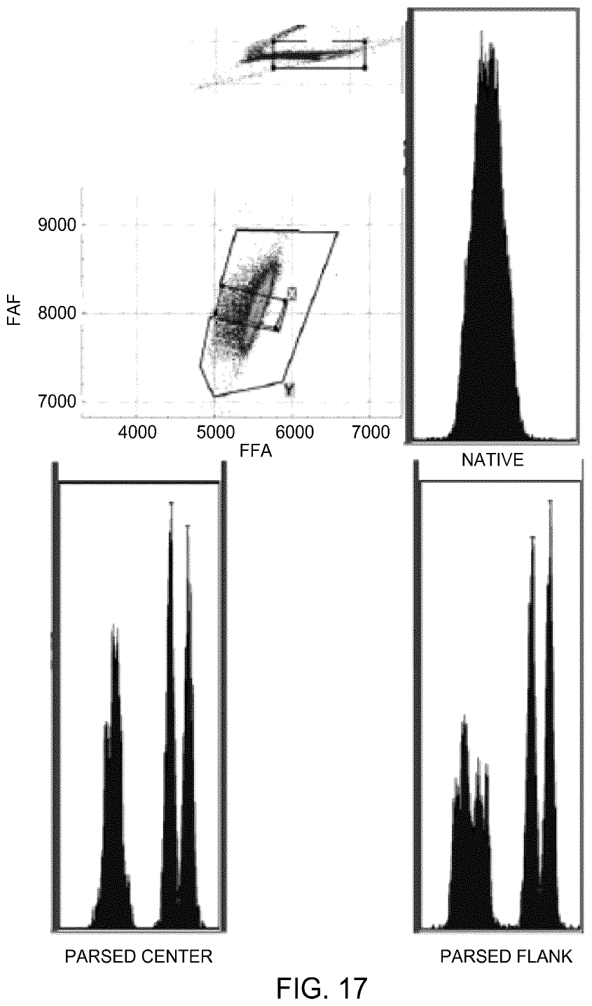

FIG. 17 depicts the parse-sorted analysis of a Boar with a Category III Stable Reciprocal Translocation confirmed by Karyotype, with aberrant DNA content where the reanalysis includes bovine stained nuclei as described above for FIG. 9.

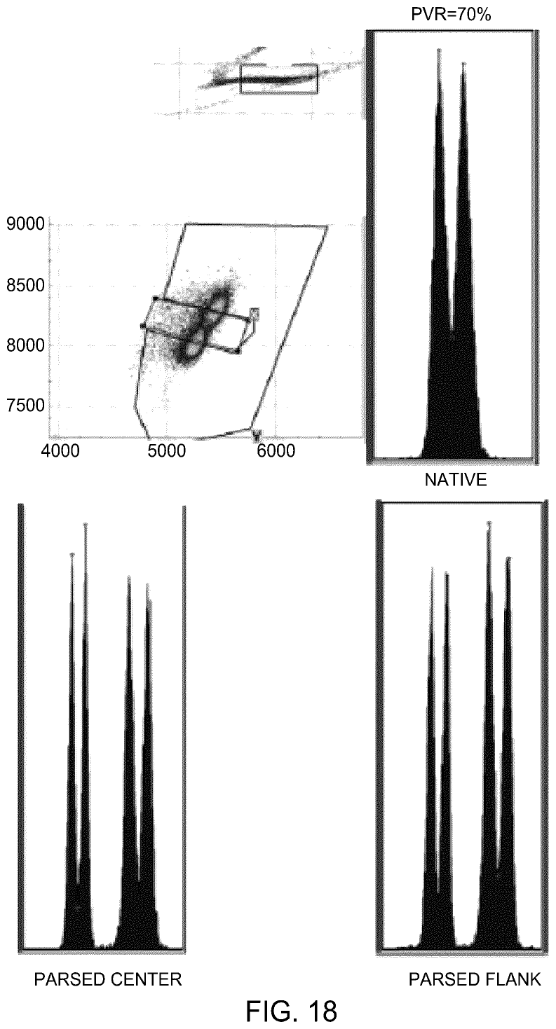

FIG. 18 depicts the parse-sorted analysis of a Boar with NORMAL DNA content where the reanalysis includes bovine stained nuclei as described above for FIG. 9.

### Example 3

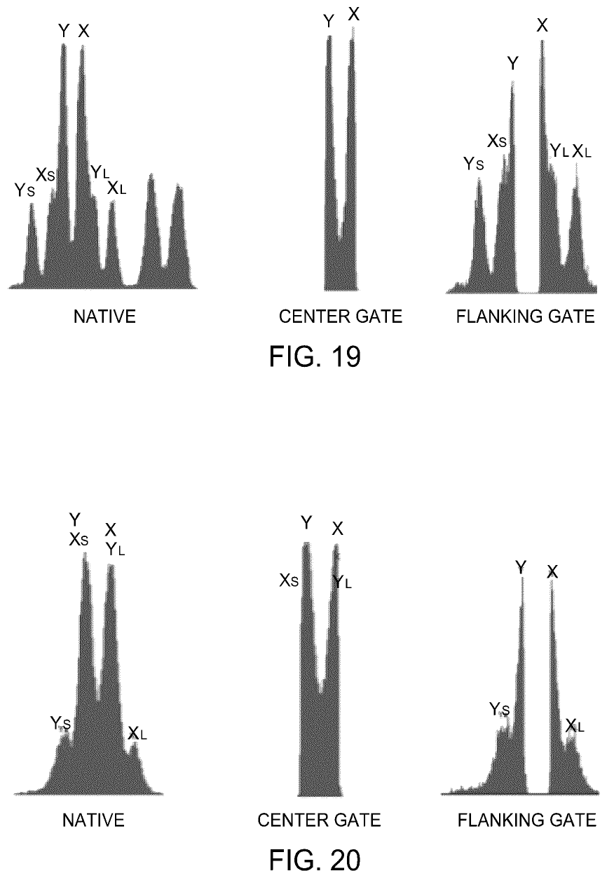

This example demonstrates the use of logical gating method on boar sperm. After collection, 400 million boar sperm cells from each of four sperm cell samples was mixed with 1 ml of a TRIS-based media comprising egg yolk and Hoechst 33342. Each sperm cell mixture was then sonicated until reaching 60° C. to create stained sperm nuclei. Each stained sperm nuclei sample was then analyzed on a flow cytometer having a forward and side fluorescence detector. Events representing oriented sperm nuclei were first gated on a multivariate plot. A histogram of fluorescence intensity for all of these gated events was generated (“native” histogram). A second multivariate plot, with forward fluorescence intensity on the y-axis and an integral of forward fluorescent intensity on the x-axis, was generated based on these gated events. A center gate and a flanking gate were placed on the second multivariate plot. Histograms of fluorescence intensities of events for each of the center gate and the flanking gate were generated. The results are shown in FIGS. 19-22.

FIG. 19 represents the above-referenced histograms (native, center gate and flanking gate) for sperm nuclei obtained from a boar having a stable chromosomal translocation where the quantity of translocated DNA is greater than the DNA quantity difference between X and Y chromosomes. While the center gate shows two peaks, which is normal, the native and flanking gate histograms show six clearly separated peaks, indicative of a chromosomal aberration.

FIG. 20 represents the above-referenced histograms (native, center gate and flanking gate) for sperm nuclei obtained from a boar having a stable chromosomal translocation where the quantity of translocated DNA is about the same as the DNA quantity difference between X and Y chromosomes. While the center gate shows two peaks, which is normal, the histograms for the native and flanking gate show four clearly separated peaks, indicative of a DNA aberration.

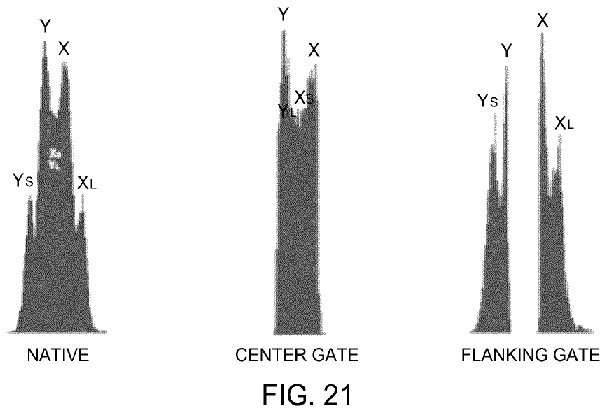

FIG. 21 represents the above-referenced histograms (native, center gate and flanking gate) for sperm nuclei obtained from a boar having a stable chromosomal translocation where the quantity of translocated DNA is less than the DNA quantity difference between X and Y chromosomes. The histograms for the native and the center gate show four poorly separated peaks and the flanking gate shows four separated peaks, all indicative of a DNA aberration.

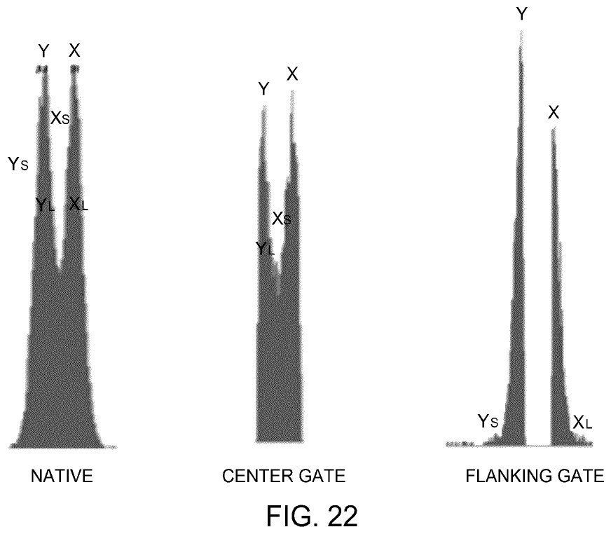

FIG. 22 represents the above-referenced histograms (native, center and flanking) for sperm nuclei obtained from a boar having a stable chromosomal translocation where the quantity of translocated DNA is much less than the DNA quantity difference between X and Y chromosomes. The histograms for the native and center gate show two poorly separated peaks, including having peak to valley ratios of 50% or less, which is indicative of a DNA aberration.

### Example 4

After collection, 400 million boar sperm cells from each of eleven sperm cell samples was mixed with 1 ml of a TRIS-based media comprising egg yolk and Hoechst 33342. Each sperm cell mixture was then sonicated until reaching 60° C. to create stained sperm nuclei. Each stained sperm nuclei sample was then parse sorted on a flow cytometer having a forward and side fluorescence detector. Events representing oriented sperm nuclei were first gated on a multivariate plot. A histogram of fluorescence intensity for all of these gated events was generated (“native” histogram). A second multivariate plot, with forward fluorescence intensity on the y-axis and an integral of forward fluorescent intensity on the x-axis, was generated based on these gated events. A center sort region and a flanking sort region were provided on the second multivariate plot. 2 million cells that generated events in the center sort region and 2 million cells that generated events in the flanking sort region were then separated and collected. Each subpopulation was then analyzed separately on a flow cytometer.

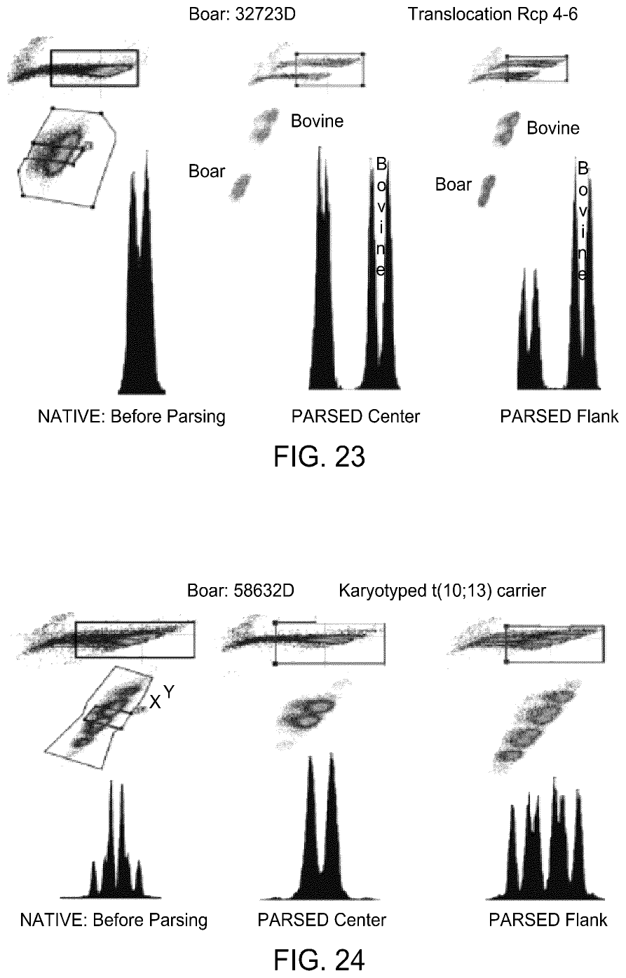

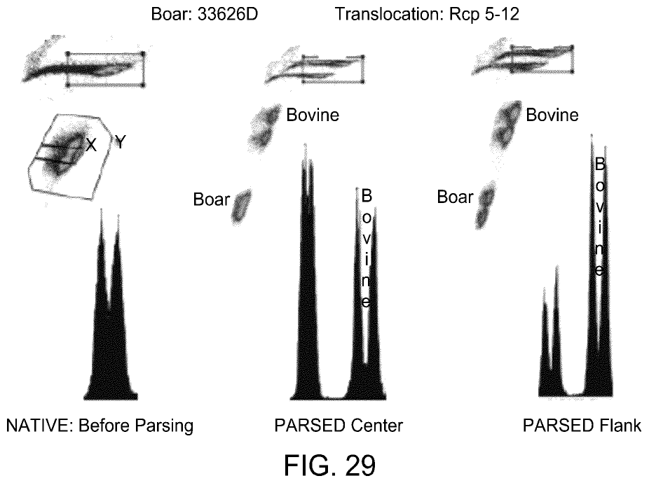

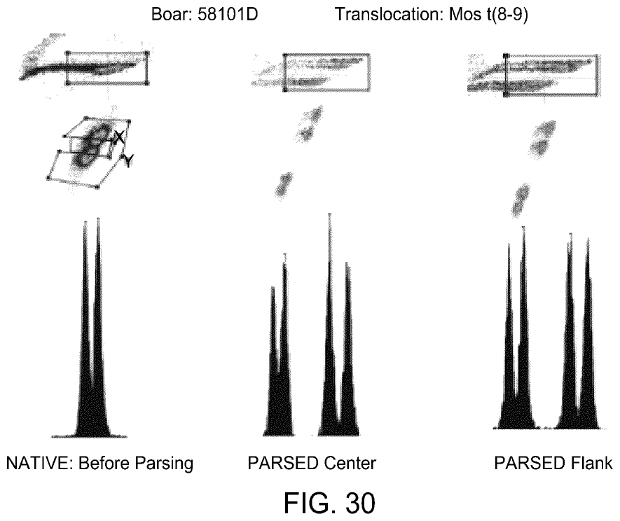

For six of the sperm nuclei samples (each sample corresponding to FIGS. 23, 29, 30, 31, 32 and 33, respectively), each collected subpopulation was first centrifuged at 950 g for 5 minutes and decanted. 1 ml of TRIS-based media comprising egg yolk, Hoechst 33342 and an internal standard comprising bovine stained sperm nuclei was added to each of the collected subpopulations from these six sperm nuclei samples. These mixtures were then sonicated and separately analyzed on a flow cytometer.

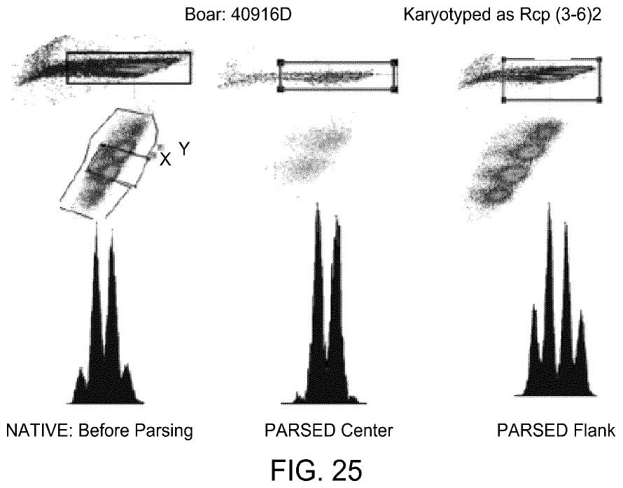

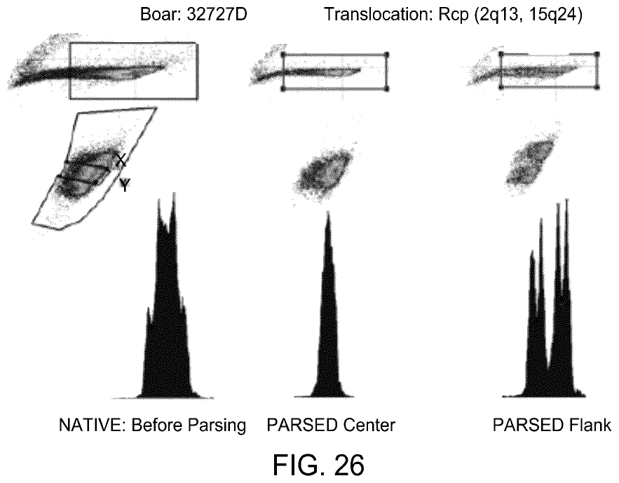

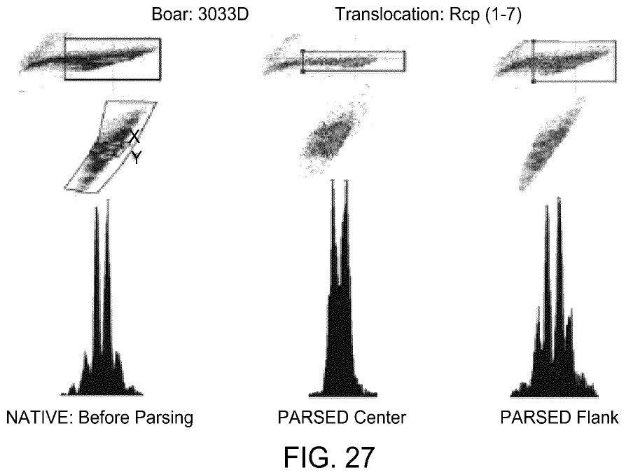

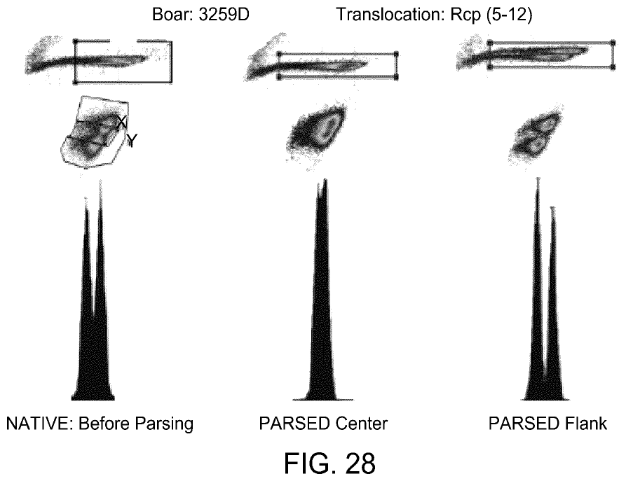

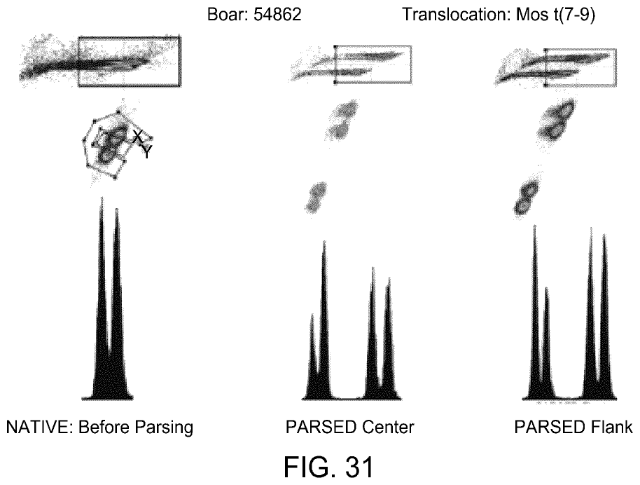

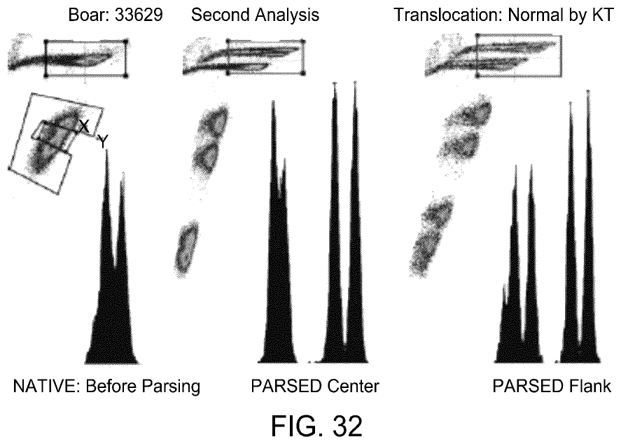

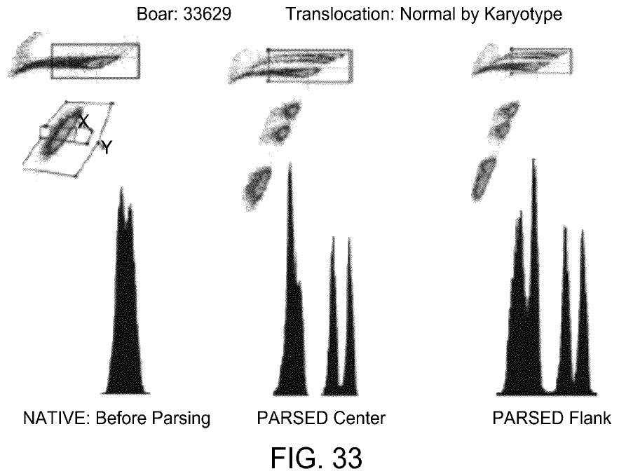

For each of the nine boars evaluated, multivariate plots and histograms of forward fluorescence intensities were generated for the unsorted sperm nuclei as well as for each of the separated subpopulations that were subsequently analyzed. These results are shown in FIGS. 23-33 for each boar, respectively, and show that the evaluated boars all have DNA aberrations, either by showing more than two separate peaks or peak to valley ratios of 50% or less on the native, center and flanking histograms (FIGS. 23-30, 32 and 33), or by showing a peak to valley ratio markedly worse than that generated for the internal standard (see, e.g., FIG. 31). Boars whose results are shown in FIGS. 32 and 33 were actually karyotyped as normal, which indicates that the invention is more sensitive in the presence or absence of DNA aberrations than traditional karyotyping methods.

### Example 5

After collection, 400 million bull (bovine) sperm cells from a sperm cell sample were mixed with 1 ml of a TRIS-based media comprising egg yolk and Hoechst 33342. The mixture was then sonicated until reaching 60° C. to create stained sperm nuclei. The sperm nuclei sample was then parse sorted on a flow cytometer having a forward and side fluorescence detector. Events representing oriented sperm nuclei were first gated on a multivariate plot. A histogram of fluorescence intensity for all of these gated events was generated (“native” histogram). A second multivariate plot, with forward fluorescence intensity on the y-axis and an integral of forward fluorescent intensity on the x-axis, was generated based on these gated events. A center sort region and a flanking sort region were provided on the second multivariate plot. Sperm nuclei that generated events in the center sort region and in the flanking sort region were then separated and collected. Each subpopulation was then re-stained with Hoechst 33342 (each 1 million sperm nuclei subpopulation stained with 20 μl of Hoechst 33342 at a concentration of 5 mg/ml) and analyzed separately on a flow cytometer.

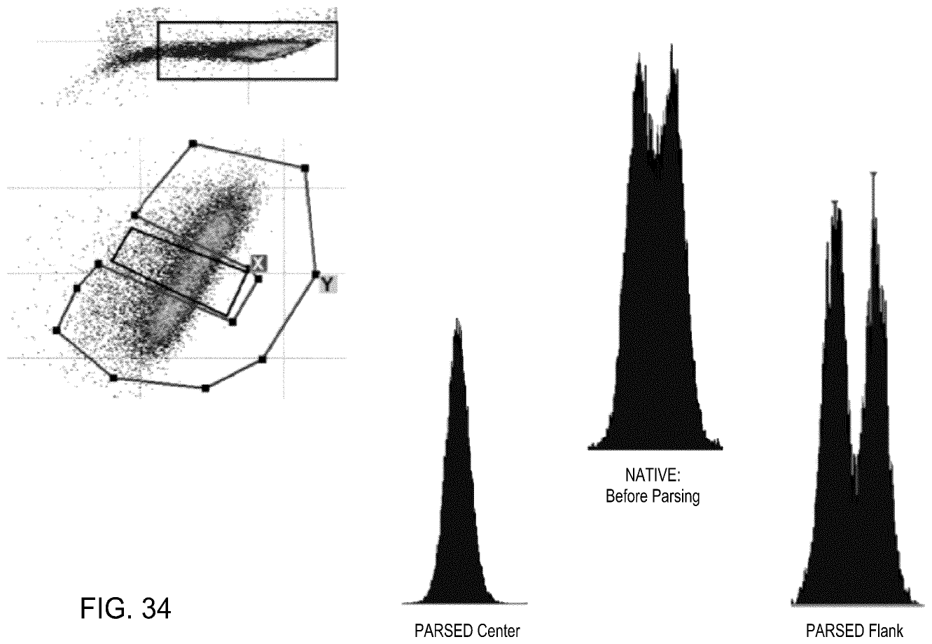

Multivariate plots and univariate histograms of forward fluorescent intensities were generated for the unsorted sperm nuclei as well as for each of the separated subpopulations that were subsequently analyzed. These results are shown in FIG. 34, and indicate that the evaluated bull has a Category IV Translocation.

### Example 6

After being collected in an extender, semen samples from 2 boars were brought to a sperm cell concentration of 200×10⁶ sperm cells/ml in test tubes. 8-10 μl of Hoechst 33342 (at a concentration of 5 mg/ml) was added for every ml of extended semen in the samples. Yellow 6 quenching dye was added at 3 μl for every ml of extended semen in the samples to identify dead or dying cells. The staining solutions were gently swirled for 10 seconds and then placed in a water bath at 34.5 to 35° C. for 60 minutes. The tubes were inverted every 15 minutes while the samples were incubating. Each staining solution was then filtered through a CellTrics® filter into properly labeled sample tubes and the stained sperm cell samples sorted via flow cytometer.

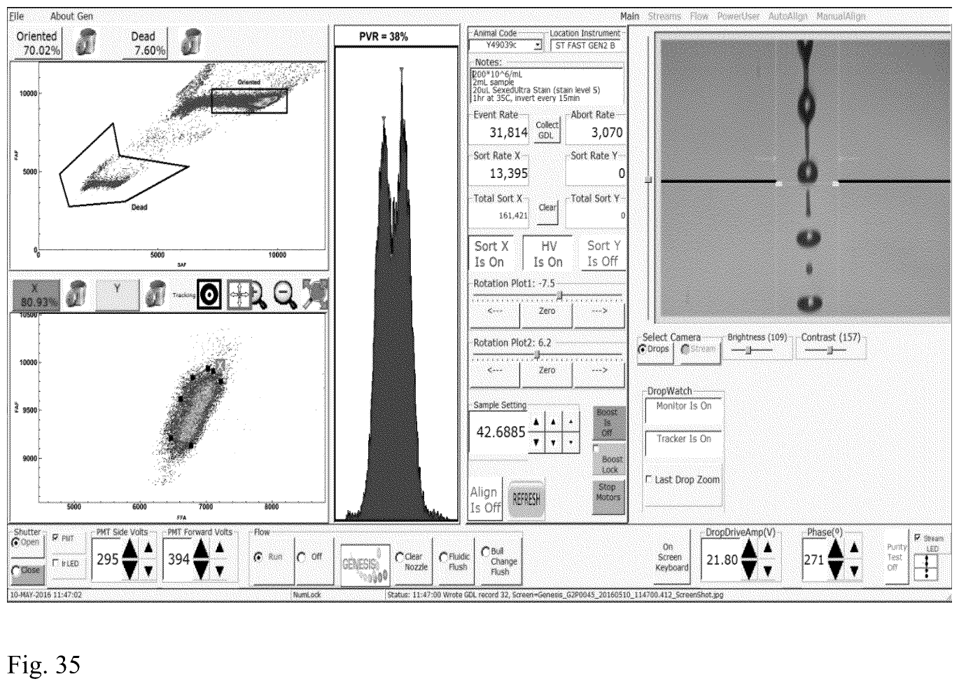

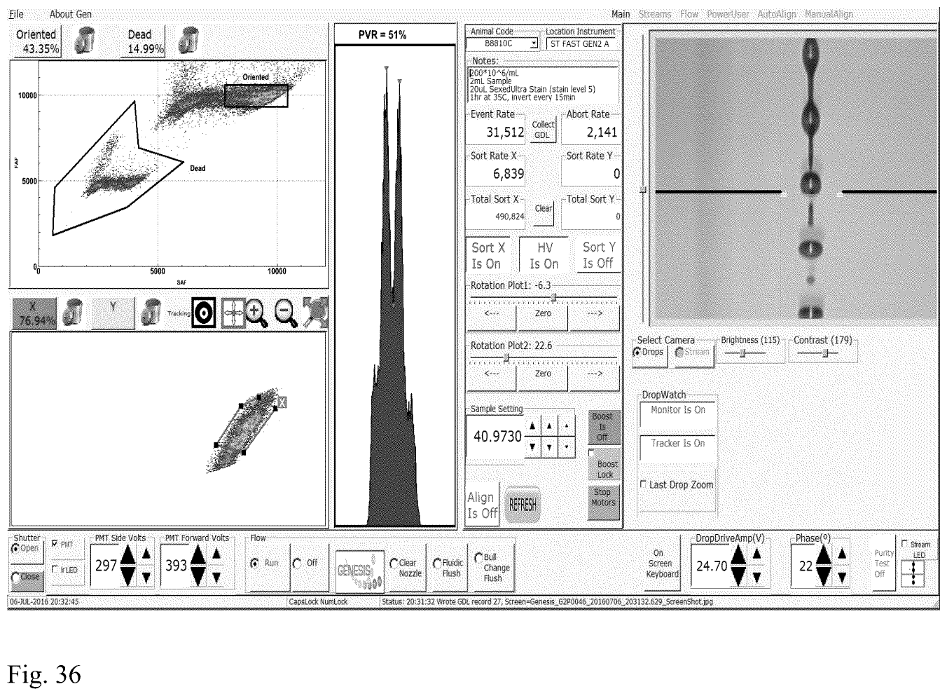

The results for each of the boars' sperm cell samples can be seen in FIGS. 35 and 36, respectively. FIG. 35 indicates that the boar has a Category IV Translocation due to the bi-modal histogram of forward fluorescence intensities having a low PVR (only 38%), while FIG. 36 indicates that the boar has a Category III Translocation based on the small side peaks present on the histogram and low PVR (only 51%).

### Example 7

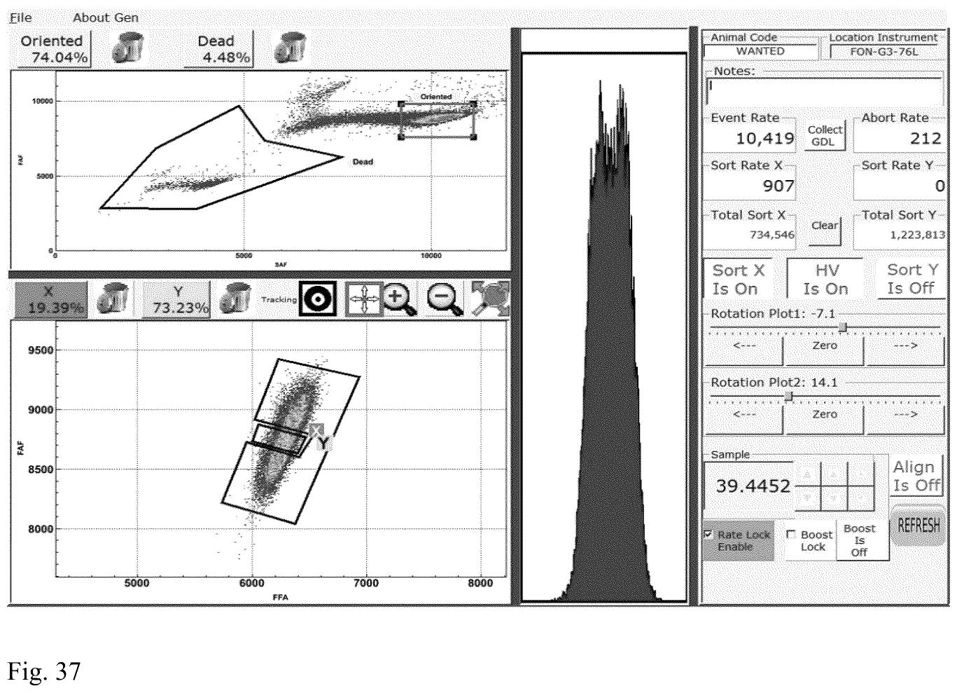

After collection, a bull (bovine)'s semen was extended and stained with Hoechst 33342 and a quenching dye using standard techniques when sex-sorting viable sperm. The stained sperm cell sample was then parse sorted on a flow cytometer having a forward and side fluorescence detector. Events representing oriented sperm nuclei were first gated on a multivariate plot, as can be seen in the upper left portion of FIG. 37. A histogram of fluorescence intensity for all of these gated events was generated as can also be seen in the middle of FIG. 37, which indicates that the bull has a Category IV Translocation due to the bi-modal histogram having a low PVR. A second multivariate plot, with forward fluorescence intensity on the y-axis and an integral of forward fluorescent intensity on the x-axis, was generated based on these gated events as can be seen on the lower left portion of FIG. 37. A center sort region and a flanking sort region were provided on the second multivariate plot as seen in FIG. 37. 1 million cells that generated events in the center sort region and 1 million cells that generated events in the flanking sort region were then separated and collected. Each subpopulation was then re-stained with Hoechst 33342 (each 1 million sperm nuclei subpopulation stained with 20 μl of Hoechst 33342 at a concentration of 5 mg/ml and 20 μl of TRIS-based media with 20% egg yolk), sonicated and then analyzed separately on a flow cytometer.

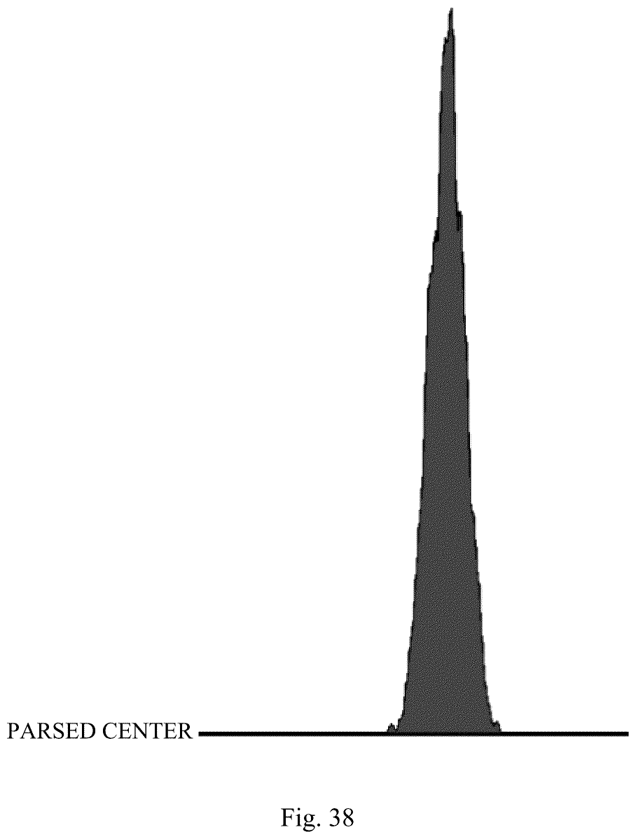

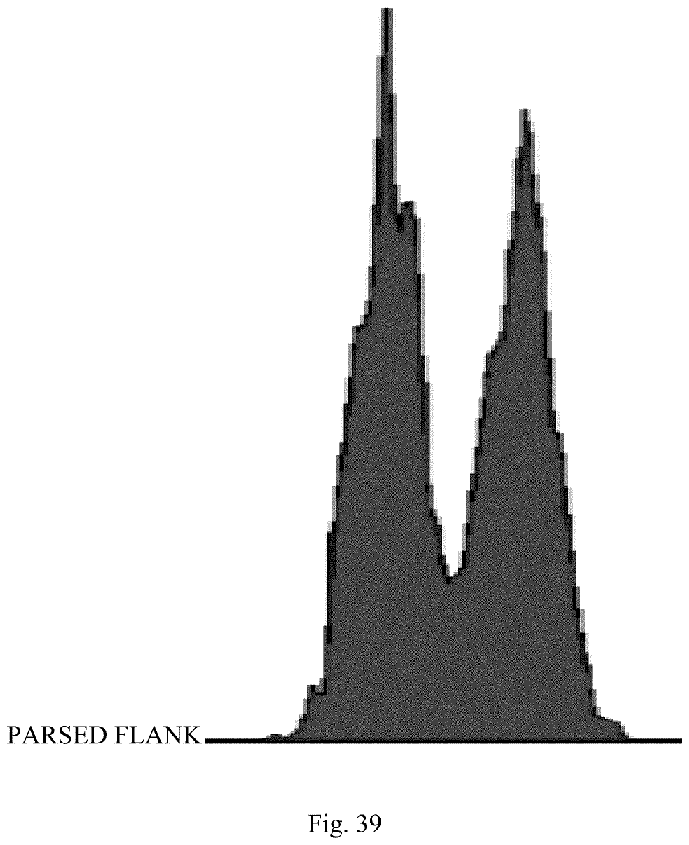

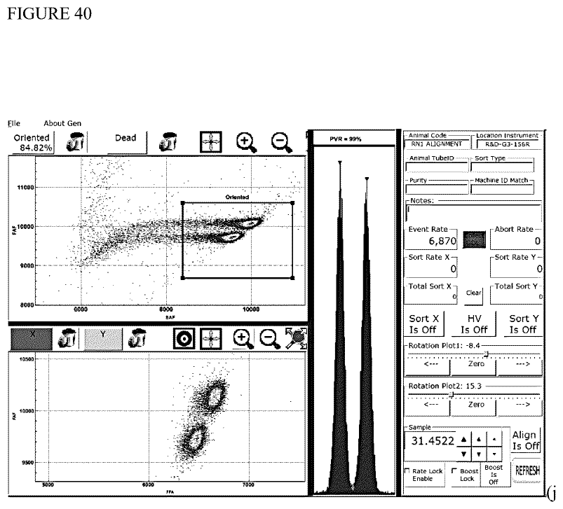

The resulting univariate histograms of forward fluorescence intensities for each subpopulation are shown in FIGS. 38 and 39 respectively, which indicate the presence of a

Category IV translocation (FIG. 38 shows a single mode (i.e. the absence of a PVR), while FIG. 39 shows a bi-modal histogram with a low PVR).

### Example 8

A flow cytometer was aligned/calibrated with high resolution, normal sperm nuclei (i.e., without a DNA aberration) stained with Hoechst 33342, which generated at least 80% oriented nuclei and a 95% PVR on the flow cytometer at an event rate of approximately 5000 events per second. FIG. 40 is a screenshot taken from the flow cytometer calibrated with normal sperm nuclei.

Sperm samples were obtained from 6 boars. 400 million sperm cells from each sperm sample were placed 1.5 ml of media comprising Hoechst 33342 and egg yolk and sonicated for 45 seconds, held for 15 seconds and then sonicated again for 30 seconds (sonicator amplitude set between 60-70%). Just prior to placing the sperm nuclei on the flow cytometer, the composition was sonicated again for 15 seconds. The stained sperm nuclei were analyzed on a flow cytometer at an event rate of approximately 1000-2000 events per second.

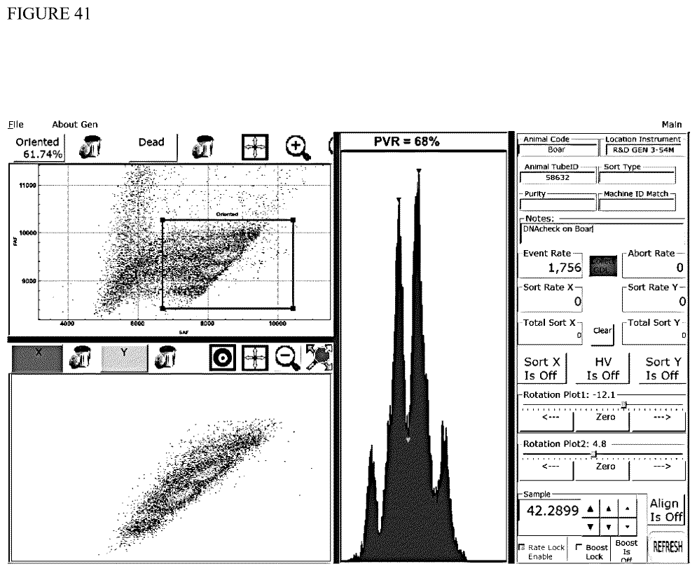

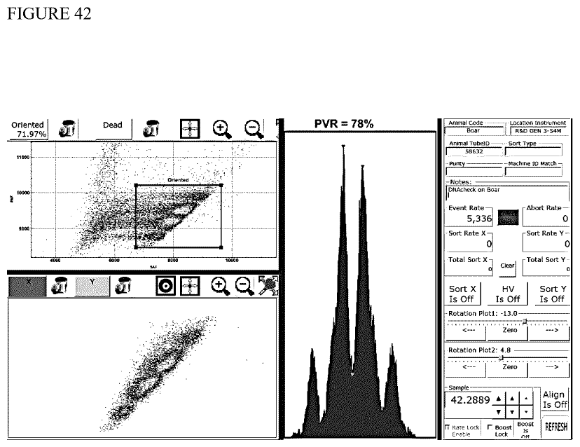

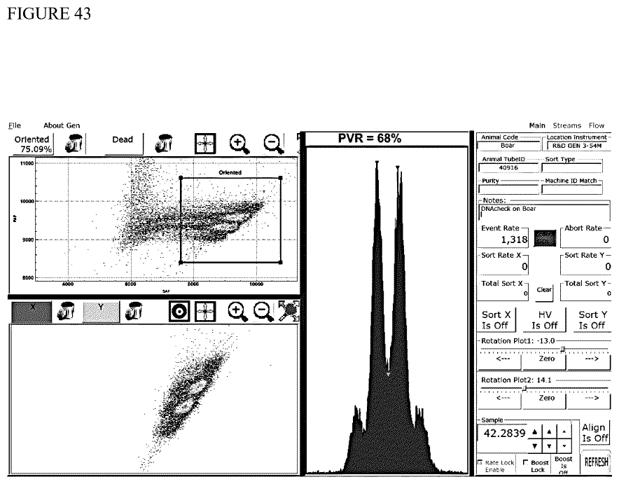

The first boar had a mos t(7-9) chromosomal translocation and a screenshot of the flow cytometer taken during the analysis of his sperm nuclei can be seen in FIG. 41. As can be seen in all three plots in FIG. 41 (i.e., a plot of forward fluorescence vs side fluorescence on the top left of FIG. 41, forward fluorescence vs an integral of forward fluorescence on the bottom left of FIG. 41 and a histogram of forward fluorescence intensities on the right of FIG. 41), at least 4 subpopulations can be seen, indicating the presence of a DNA aberration. FIG. 42 is also a screenshot of an analysis of the first boar, except that 500 μl of 20% egg yolk media was added to the sperm nuclei composition, which improved orientation and resolution.

The second boar had an rcp (3;6)_2 translocation and a screenshot of the flow cytometer taken during the analysis of his sperm nuclei (with an additional 500 μl of 20% egg yolk media added) can be seen in FIG. 43. Again, more than two subpopulations can be visualized in all of the plots shown in FIG. 43 indicating the presence of a DNA aberration.

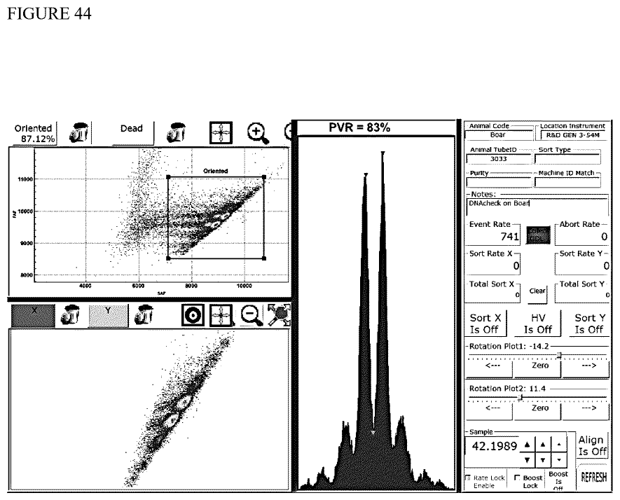

The third boar had an rcp_1-7 translocation and a screenshot of the flow cytometer taken during the analysis of his sperm nuclei (with an additional 750 μl of 20% egg yolk media added) can be seen in FIG. 44. More than two subpopulations can be visualized in all of the plots shown in FIG. 44, indicating the presence of a DNA aberration.

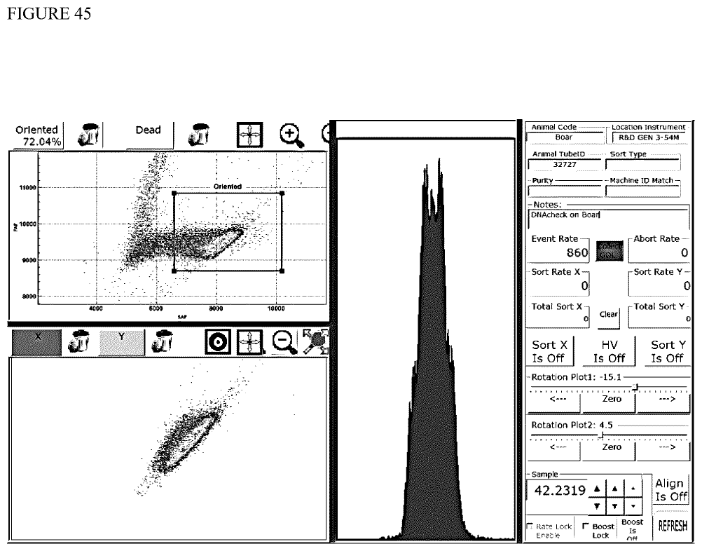

The fourth boar had an rcp (2q13,15q24) translocation and a screenshot of the flow cytometer taken during the analysis of his sperm nuclei can be seen in FIG. 45. While at least two subpopulations can be seen FIG. 45, the subpopulations are indistinct and appear to be almost entirely merged with one another in all three plots, indicating the presence of a DNA aberration, in particular exhibiting a very poor PVR in the histogram of forward fluorescence intensities and the presence of a third peak or subpopulation in between the two peaks or subpopulations normally associated with X-bearing and Y-bearing sperm.

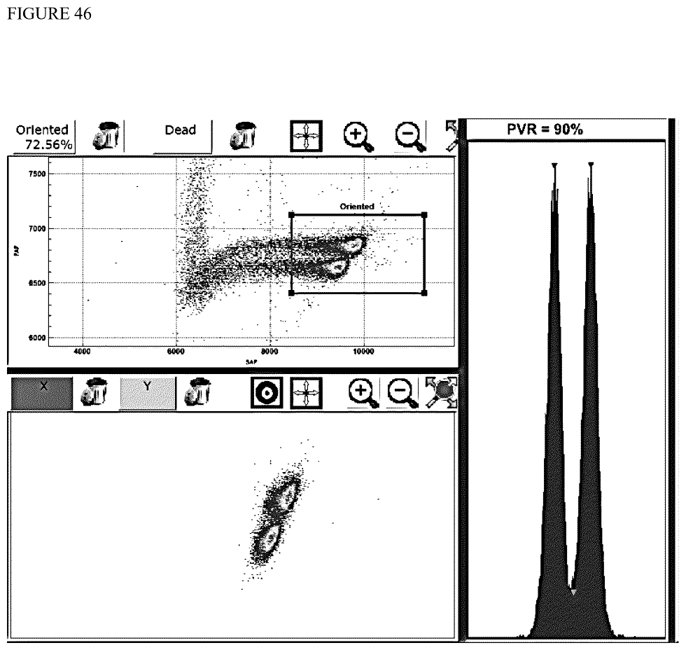

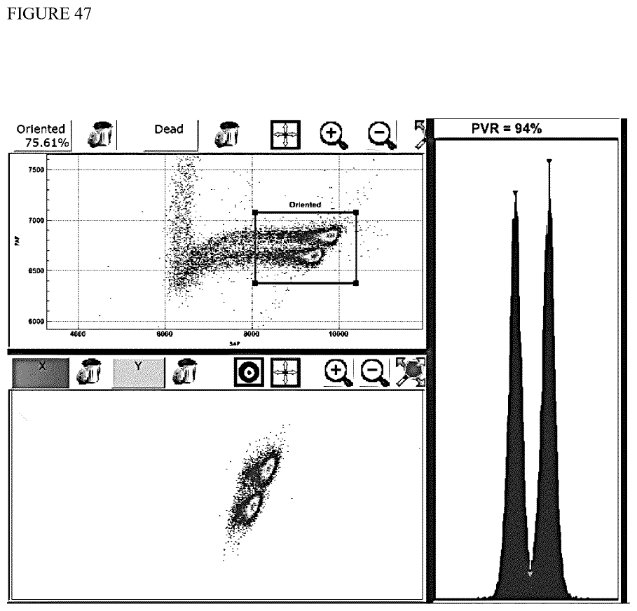

The fifth and sixth boars had normal karyotypes and as can be seen in FIGS. 46 and 47 respectively, their plots showed only two distinct subpopulations, indicating the absence of a DNA aberration.

### Example 9

Bovine X bearing sperm cells have typically 3.8% more fluorescence signal than Y bearing sperm cells. Considering the Bos taurus (domestic cattle) genome assembly ARS-UCDv1.2, the genome sizes of X and Y bearing haploid cells are expected to be 2,628,394,923 bp and 2,532,685,960 bp, respectively, which translates into a differential of 3.6%. The 0.2% nominal difference between fluorescence-based and sequence-based DNA content estimates could be explained by additional X and Y sequences that are not included in the main assembly since 2,180 unassembled scaffolds in ARS-UCDv1.2 amount to over 87 Mbp. Therefore, assuming interchangeability of fluorescence estimates with base pair content, we simulated a range of scenarios to determine the behavior of 0° fluorescence density plots in the presence of reciprocal balanced translocations (bRCT).

The variation in 0° fluorescence (i.e., forward fluorescence or DNA content) were modeled as a mixture of scaled t distributions in R v4.1.1. For each subpopulation p of sperm cells, nₚ observations were first sampled from a t distribution with υₚ degrees of freedom. Then, observations were multiplied by a standard deviation of σₚ and summed to a mean of μₚ, both expressed in Mbp. Based on empirical values from sperm sorting, the following parameter values were adopted: υᵢ=10 and σᵢ=25. Considering that sexed semen is packaged in doses of 4 million cells, the number of observations per subpopulation was set to nₚ=2×4×10⁶, where P is the total number of subpopulations. For example, simulation of normal semen implied P=2, μₓ=2628.394923, μ_{y}=2532.685960, and nₓ=n_{y}=4×10⁶. To simulate bRCT, translocated autosomes were sampled at random, with breakpoints sampled in each chromosome from a uniform distribution with minimum and maximum parameters set to 1 and chromosome size, respectively. Eight different subpopulations were assumed in this case, namely X, XS, XL, XLS, Y, YS, YL and YLS. The mean of each subpopulation was determined by summing up the lengths of their respective haploid genomes.

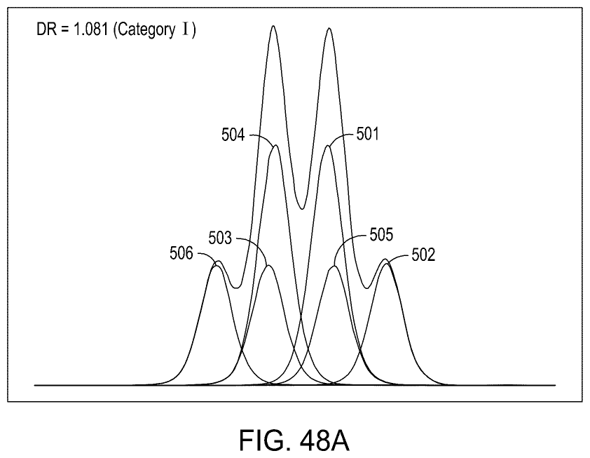

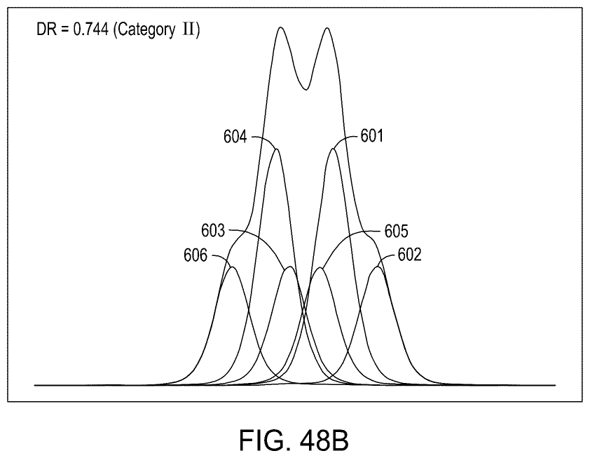

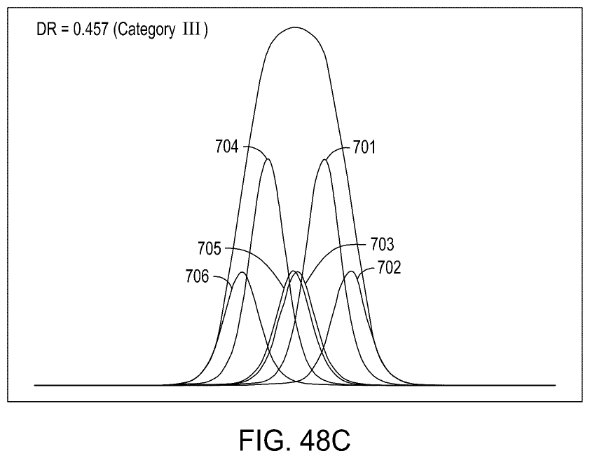

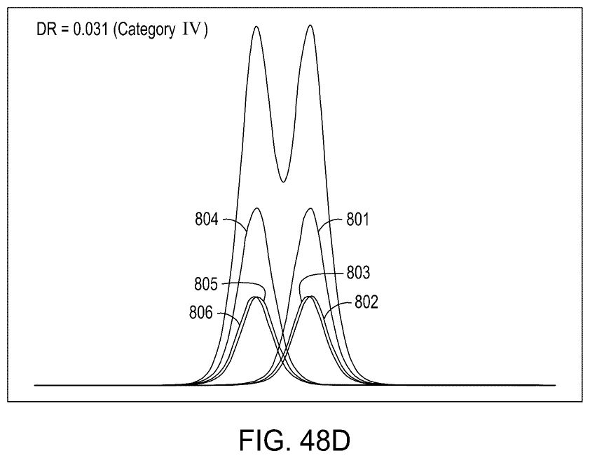

Our simulation of bRCT resulted in four distinctive fluorescence density patterns corresponding to the Categories delineated in Example 1 above (see FIGS. 48A to 48D, corresponding to Categories I to IV, respectively). Referring to FIG. 48A, subpopulations X+XLS 501, XS 503, XL 502, Y+YLS 504, YS 506 and YL 505 are indicated for the first pattern, Category I, which was characterized by the presence of accessory modes, with the leftmost mode representing Y bearing cells carrying the smallest derivative chromosome (YS 506) and the rightmost mode representing X bearing cells carrying the largest derivative chromosome (XL 502). By computing the ratio between the (XL−X)/XL and (X−Y)/X mass differentials, hereafter referred to as the “DR” statistic, we found that Category I translocations had DR typically ranging from 0.9 to 1.4. Referring to FIG. 48B, subpopulations X+XLS 601, XS 603, XL 602, Y+YLS 604, YS 606 and YL 605 are indicated for the second pattern, Category II, which still presented visible accessory modes, but the valleys between the YS 606 and Y+YLS 604 modes and between the X+XLS 601 and XL 602 modes varied from very shallow to nearly flat, and the valley between X+XLS 601 and Y+YLS 604 modes was further shallower than those observed in Category I plots. For Category II translocations, DR ranged from 0.6 to 0.9. Referring to FIG. 48C, subpopulations X+XLS 701, XS 703, XL 702, Y+YLS 704, YS 706 and YL 705 are indicated for the third pattern, Category III, which comprised nearly or completely unimodal distributions with 0.3<DR≤0.6. Referring to FIG. 48D, subpopulations X+XLS 801, XS 803, XL 802, Y+YLS 804, YS 806 and YL 805 are indicated for the last pattern, Category IV, which presented bimodality but with a smaller peak-to-valley ratio (PVR) as compared to normal XY density plots, with DR values typically below 0.3.

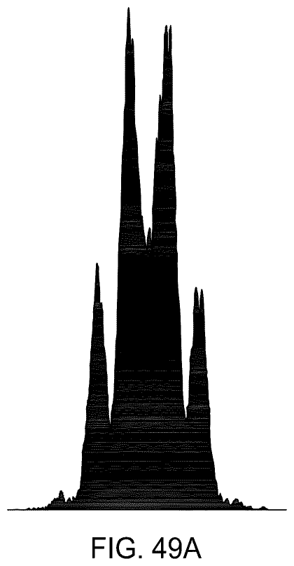

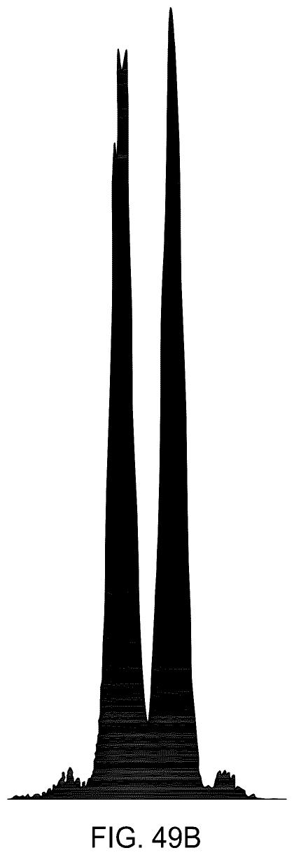

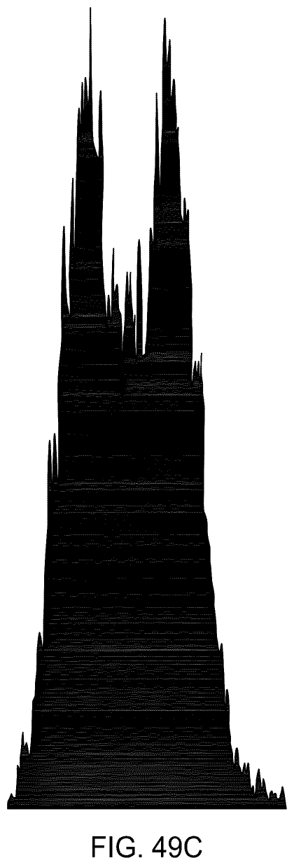

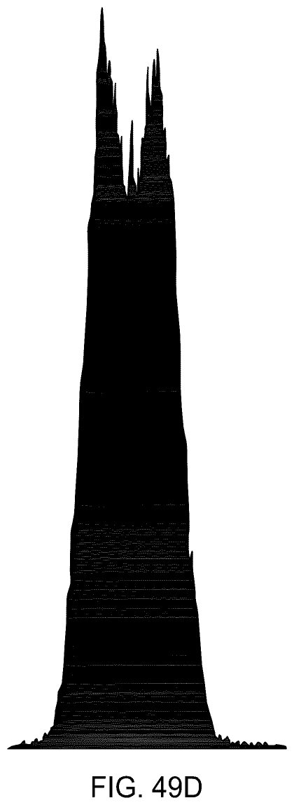

With density plot patterns for bRCT determined, semen samples from 668 AI bulls was collected for flow-cytometric analysis. After staining, samples were analyzed via flow cytometer. After gating for live and oriented cells, a univariate plot of forward fluorescence values was generated for each sample. Four cases of bRCT were detected based on these univariate plots, including two Category I cases (see FIGS. 49A and 49B), one Category III case (see FIG. 49C) and one Category IV case (see FIG. 49D). One of the Category I translocations had lower than expected peaks for YS and XL cells, which suggests that a lower proportion of these cells could be retained for analysis after gating oriented live cells. Based on the obtained data, the prevalence of bRCT in AI bulls was estimated at 0.60% (1 every 167) across all breeds. Since the distribution of breeds in the data was highly skewed toward Holstein cattle, a prevalence of 0.70% (1 every 143) considering only Holstein samples may be a less biased estimate.

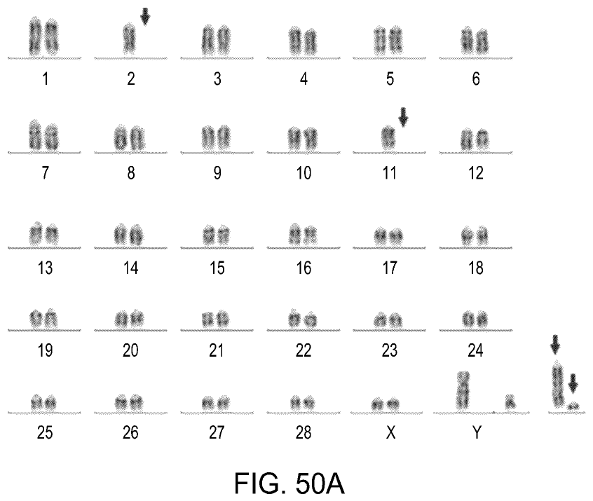

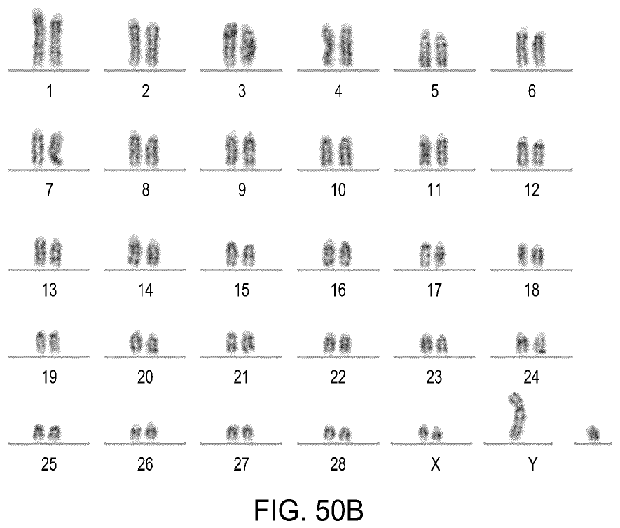

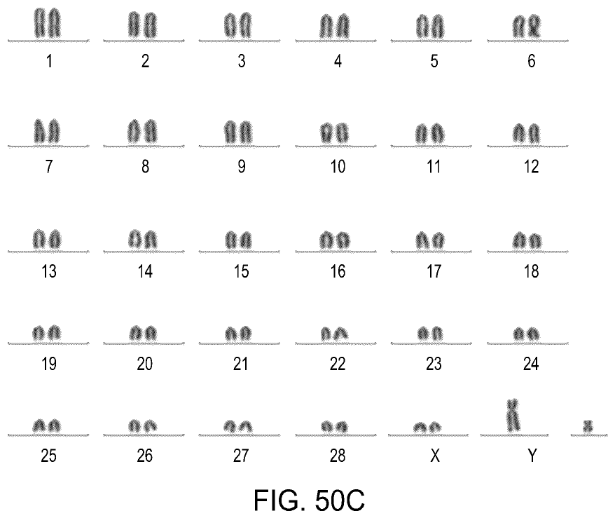

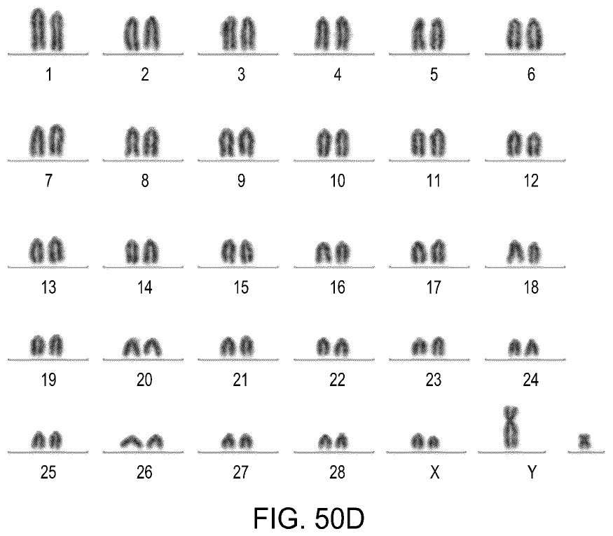

Blood samples from the animals that tested positive for bRCT by the above method were sent for G-banding karyotyping. All karyotype results returned normal (see FIGS. 50B, 50C and 50D) except for one of the bulls carrying a Category I translocation (see FIG. 50A). The latter was described as having two derivative chromosomes, with the first being a large acrocentric chromosome likely resulting from a tandem fusion of chromosome 2 with chromosome 11, and the second deriving from the terminal end of chromosome 11. These findings indicated that karyotyping was limited to the detection of translocations resulting in very large or very small derivative chromosomes, which reflects its dependency on visual inspection of chromosomes. In contrast, flow cytometric analysis by the above method had a higher sensitivity and detected cases involving varied sizes of derivative chromosomes.

### Example 10

The following Example demonstrates that sequencing of sorted sperm cells can confirm translocations. As noted above, in routine sperm sex-sorting, the scatterplot of 0° and 90° fluorescence values is used to select subpopulations of oriented live cells for sorting, such that the flow cytometer can be set to positively, neutrally or negatively charge droplets that will be separated by electrostatically charged plates and directed to collecting receptacles. For example, droplets containing Y bearing sperm cells can be positively charged, droplets containing X bearing sperm cells can be negatively charged, and droplets containing non-oriented or dead sperm cells can be kept neutral to go to waste. This procedure was adapted to obtain subpopulations enriched for YS or XL cells by selecting the upper and lower tails of the density distributions in bRCT cases. A subpopulation comprising XL cells can be obtained by placing a sort gate on a univariate or bivariate plot in which the gate encompasses events in the 84^{th} percentile or higher in terms of forward fluorescence intensity (i.e., DNA content). Additionally, a subpopulation comprising YS cells can be obtained by placing a sort gate on a univariate or bivariate plot in which the gate encompasses events in the 16^{th} percentile or lower in terms of forward fluorescence intensity (i.e., DNA content). The separated upper and lower tail subpopulations from each sample were then subjected to DNA extraction and short read sequencing at about 30× coverage.

Paired-end libraries were produced for the upper and lower tail parsed samples and sequenced at 30× coverage using S2 flow cells in the Illumina® NovaSeq platform. The ARS-UCDv1.2 Bos taurus reference genome augmented with the Btau 5.1 Y chromosome assembly was used for read mapping with the Burrows-Wheeler Alignment (BWA) software v0.7.17-r1198-dirty. Picard tools v3.2.0 was used to mark duplicated reads, and samtools v1.20 was used for read sorting and indexing. Coverages of non-overlapping windows of 50 kbp were calculated for each aligned bam file using mosdepth 0.3.8. For each bull, coverage data from upper and lower tail parsed samples were further subtracted from each other and then scaled by subtracting the mean and dividing by the standard deviation of coverage differences.

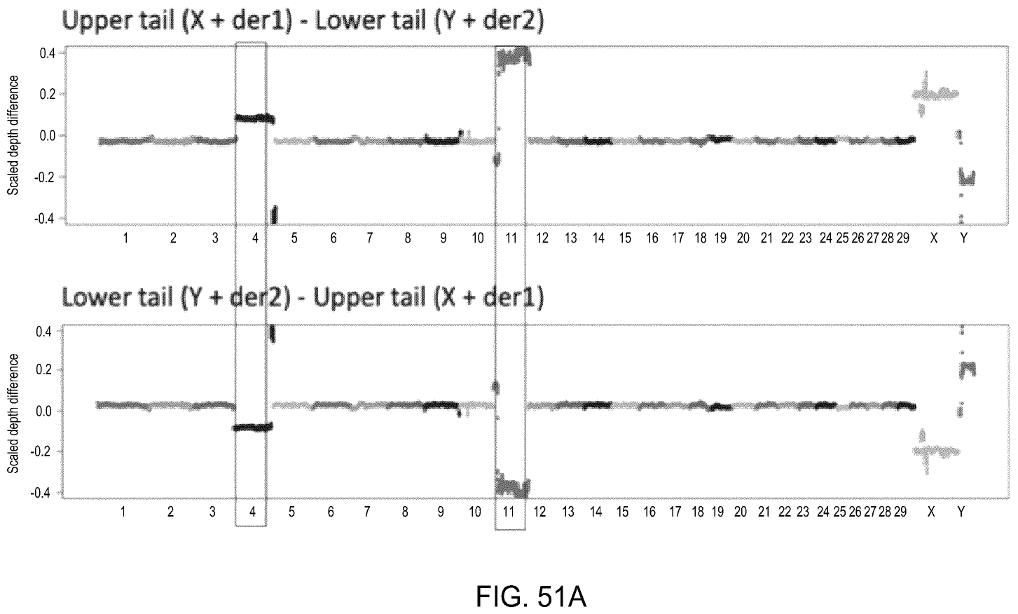

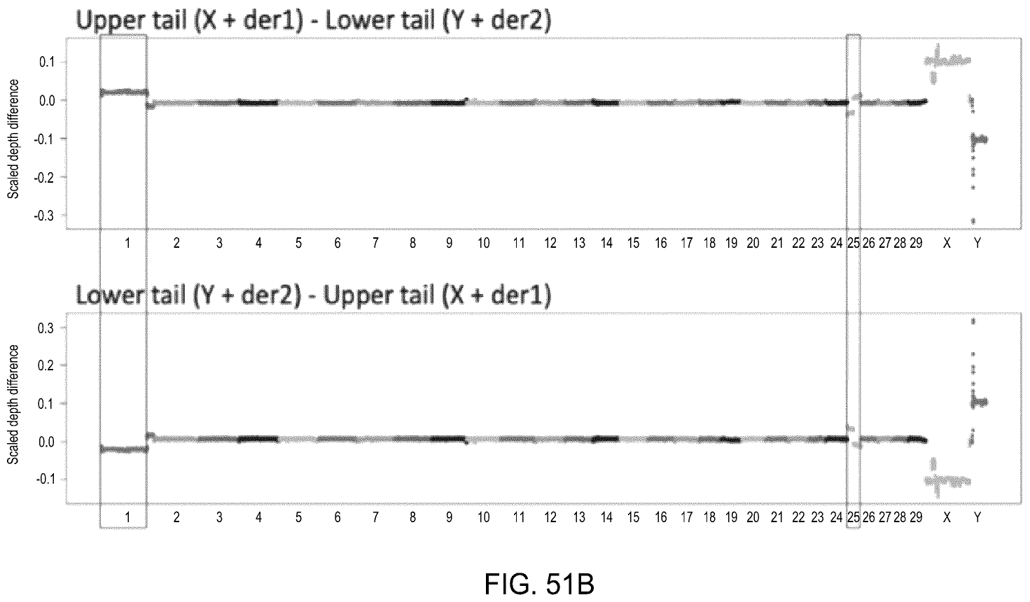

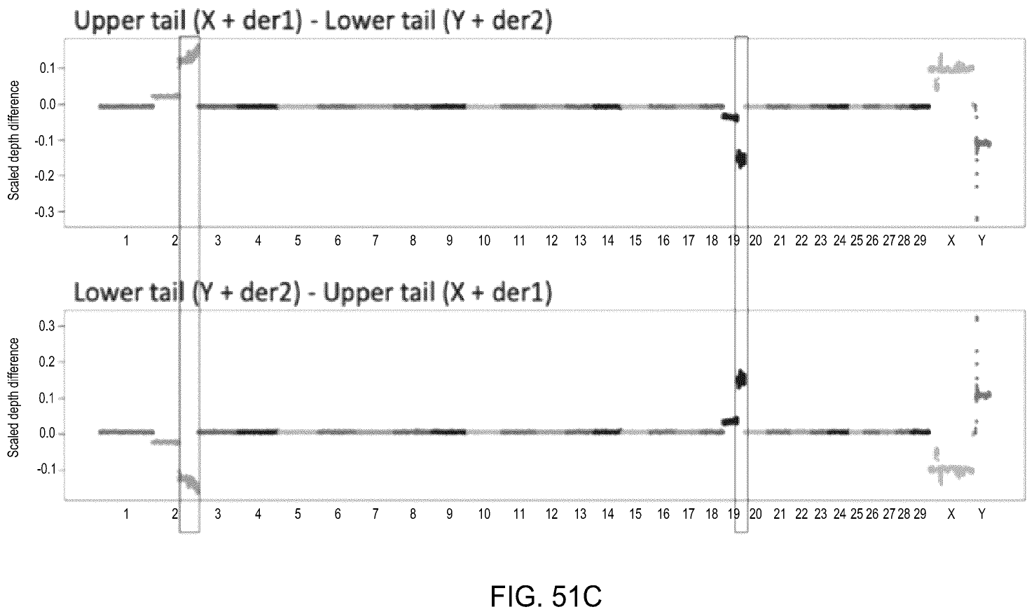

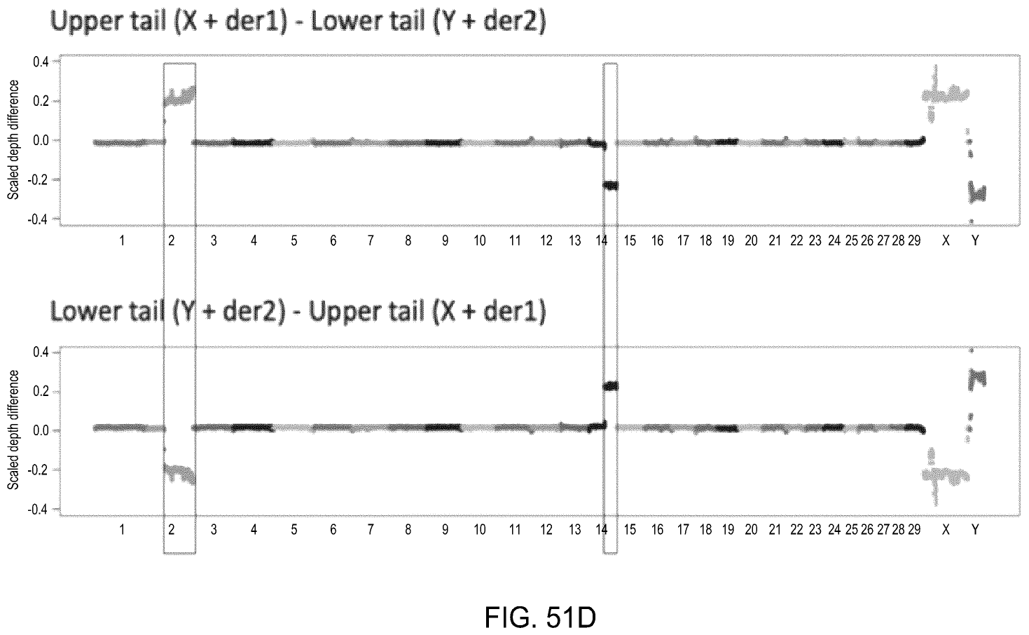

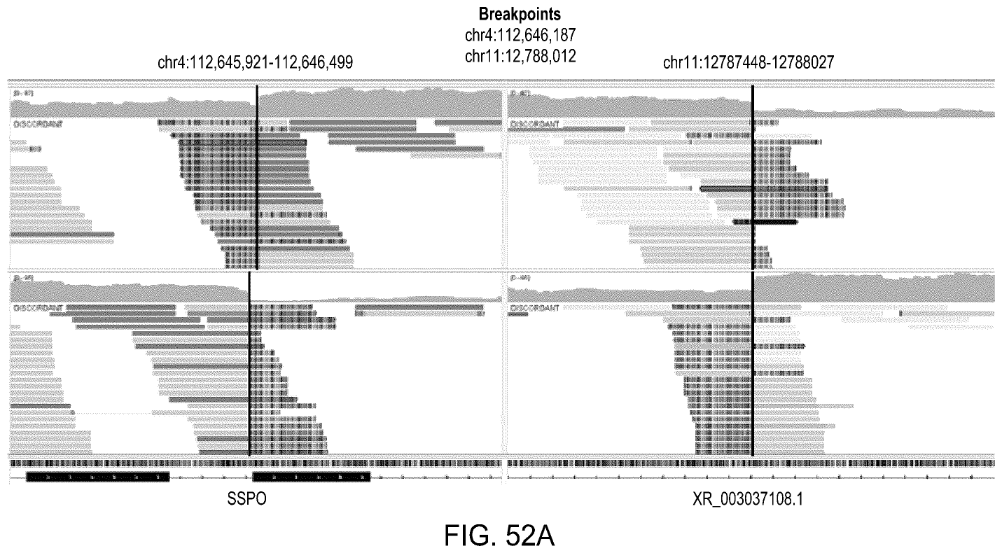

After aligning the paired end reads to the reference genome, base sequencing depth was averaged across non-overlapping windows of 50 kbp for the upper and lower tail samples separately. The scaled sequencing depth difference between the two tails was then computed to reveal the chromosomes involved in each bRCT event (see FIGS. 51A to 51D, each corresponding to FIGS. 49A to 49D and 50A to 50D, respectively). In all cases, the affected chromosomes were readily apparent. Interestingly, the Category I case suggested as a translocation of chromosomes 2 and 11 by G-banding karyotyping was identified instead as t(4;11) through sequencing (see FIG. 51A). The other Category I case, and the Category III and Category IV cases were identified as t(1;25), t(2;19) and t(2;14) translocations, respectively (see FIGS. 51B to 51D, respectively). All translocations were further validated through fluorescence in situ hybridization (FISH).

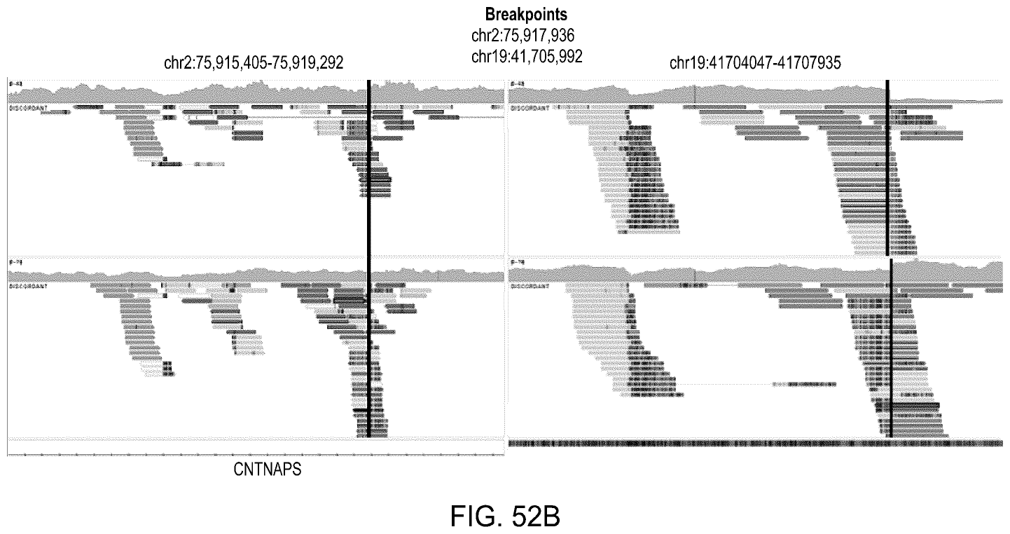

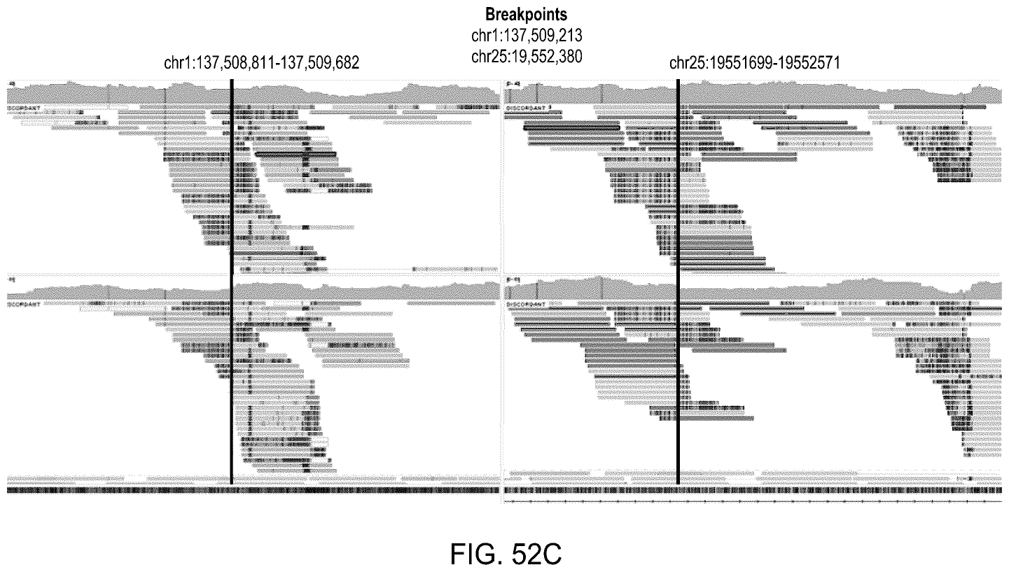

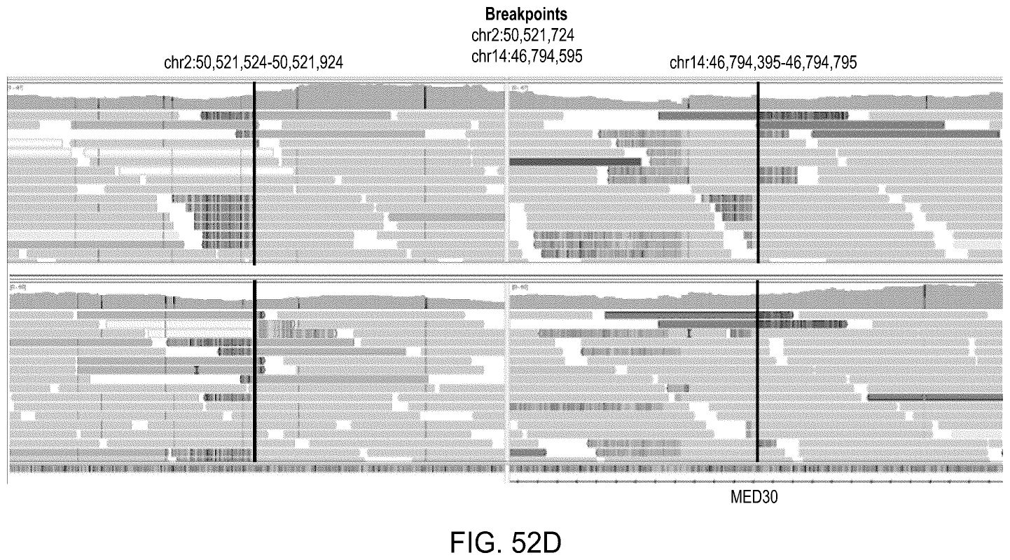

By applying a Hidden Markov Model (HMM) to the scaled difference in sequencing depth between the upper and lower tail samples, the translocation breakpoints were mapped to single pairs of candidate regions of 100 kbp or shorter. Re-sampling reads to artificially reduce coverage from 30× to as low as 0.5× did not significantly impact HMM mapping, indicating that low pass, skim sequencing could be used to validate cases where the identities of affected chromosomes are required but exact breakpoint positions are not. Inspection of candidate regions in Integrative Genomic Viewer (IGV) further resolved breakpoints to single base pair positions (see FIGS. 52A to 52D, each corresponding to FIGS. 49A to 49D, 50A to 50D and 51A to 51D, respectively). Using the resolved breakpoints, the lengths of the derivative chromosomes and the resulting XL−YS differentials were estimated, revealing that these differentials could be predicting with high accuracy using flow-cytometric analysis.

Candidate regions harboring translocation breakpoints were initially mapped using a Hidden Markov Model (HMM) built with the HMM v1.0.1 package in R. The model included three hidden states representing copy loss, neutral and gain, with transition probabilities of 10% between states and 90% within states, and emission probabilities of 50% within states and 25% between states. The input data to the HMM comprised a transformation of the scaled depth differences where the values were first smoothed by taking the median across 10 neighboring windows (˜500 kbp), and then converted to the following emission values: −1 if the smoothed value was at least one standard unit below the mean, +1 if the smoothed value was at least one standard unit above the mean, and 0 otherwise. Runs of equally classified states were then aggregated to report coordinates and sizes of segments. Typically, non-translocated chromosomes appeared as whole chromosome-sized neutral segments, X appeared as copy gain and Y as copy loss. Chromosomes implied in translocations appeared as two segments each, one copy gain and one copy loss, with the transition position between gain and loss corresponding to approximate breakpoint positions.

Candidate regions were further refined by visually inspecting upper and lower tail alignments with the Integrative Genomics Viewer (IGV) v2.13.2. Visualization of reads was set to “view as pairs” and then reads were grouped either by “reference concordance” or by “chromosome of mate.” Candidate regions were screened for enrichment of reads with paired ends mapping to a different chromosome. The exact breakpoint positions were determined by running the BLAT tool with soft-clipped bases in split reads.

What we claim is:

1. A method of identifying a translocation in a male comprising:
providing a first subpopulation and a second subpopulation of sperm cells from a population of sperm cells from the male, wherein the first subpopulation comprises the 84^{th} percentile or higher of sperm cells in the population with respect to detected DNA quantity and the second subpopulation comprises the 16^{th} percentile or lower of sperm cells in the population with respect to detected DNA quantity;
sequencing DNA from the first subpopulation and the second subpopulation;
generating reads for the first subpopulation and reads for the second subpopulation;
aligning the reads for the first subpopulation and the reads for the second subpopulation to a reference genome;
averaging the base sequencing depth across non-overlapping windows for the first subpopulation and for the second subpopulation;
computing a scaled sequencing depth difference between the first subpopulation and the second subpopulation; and
identifying a translocation in the male based on the scaled sequencing depth difference.

2. The method of claim 1, wherein sequencing comprises short read sequencing.

3. The method of claim 1, wherein the step of sequencing is performed at a depth of 10× or lower.

4. The method of claim 1, further comprising the step of mapping translocation breakpoints to single pairs of regions within one or more chromosomes using the scaled sequencing depth difference.

5. The method of claim 4, wherein the regions are 100 kbp in length or shorter.

6. The method of claim 4, wherein the step of sequencing is performed at a depth of 10× or higher.

7. The method of claim 1, wherein the detected DNA quantity is detected by a flow cytometer.

8. A method of processing sperm cells comprising:
staining a population of sperm cells with a DNA-selective dye;
entraining the population in sheath fluid;
exposing the entrained population to electromagnetic radiation to generate events for the population;
gating a first subpopulation of the events, wherein each event within the first subpopulation corresponds to a sperm cell in the 84^{th} percentile or higher of sperm cells in the population with respect to detected DNA quantity;
gating a second subpopulation of the events, wherein each event within the second subpopulation corresponds to a sperm cell in the 16^{th} percentile or lower of sperm cells in the population with respect to detected DNA quantity;
separating the sperm cells corresponding to the gated first subpopulation from the population of sperm cells;
separating the sperm cells corresponding to the gated second subpopulation from the population of sperm cells; and
genotyping DNA from the separated sperm cells corresponding to the gated first subpopulation and DNA from the separated sperm cells corresponding to the gated second subpopulation.
